(19) United States
(12) Patent Application Publication
(10) Pub. No.: US 2019/0117667 A1
(43) Pub. Date: Apr. 25, 2019

(54) ANTICANCER ACTIVITY OF PLATINUM(II) TETRASELENONE COMPLEXES

(71) Applicant: King Fahd University of Petroleum and Minerals, Dhahran (SA)

(72) Inventors: Ali Osman S. ALTOUM, Dhahran (SA); Muhammad ALTAF, Dhahran (SA); Anvarhusein A. ISAB, Dhahran (SA)

(73) Assignee: King Fahd University of Petroleum and Minerals, Dhahran (SA)

(21) Appl. No.: 15/791,805

(22) Filed: Oct. 24, 2017

(51) Int. Cl. A61K 31/555; C07F 15/00; A61K 45/06

(52) U.S. Cl. CPC A61K 31/555; A61K 45/06; C07F 15/0093

(57) ABSTRACT

A method for treating a proliferative disease, disorder, or condition comprising administering a Pt(II) tetra-selenone complex. A Pt(II)-tetra-selenone complex.

### BACKGROUND

### Field of the Invention

The present disclosure relates to Pt(II)-tetra-selenone complexes with selenones as described herein (“HLn”), such as those with general formulae [Pt(HLn)₄]Cl₂, and to a method for treating or inhibiting cancer using them.

### Description of Related Art

The “background” description provided herein is for the purpose of generally presenting the context of the disclosure. Work of the presently named inventor(s), to the extent it is described in this background section, as well as aspects of the description which may not otherwise qualify as prior art at the time of filing, are neither expressly or impliedly admitted as prior art against the present invention.

Platinum-based anticancer agents are widely used as first-line drugs in cancer chemotherapy for various solid tumors, such as testicular, ovarian, bladder, head and neck, and lung cancers. Cisplatin is particularly effective in the treatment of testicular cancer with a cure rate of over 90% and nearly 100% when tumors are discovered early. See T. C. Johnstone, K. Suntharalingam, S. J. Lippard. Chem. Rev., 2016, 116, 3436-3486; S. Medici, M. Peana, V. M. Nurchi, J. I. Lachowicz, G. Crisponi, M. A. Zoroddu, Coord. Chem. Rev., 2015, 284, 329-350; E. Shaili, Sci. Prog., 2014, 97, 20-40; X. Wang, Z. Guo. Chem. Soc. Rev., 2013, 42, 202-224; Y. Jung, S. J. Lippard. Chem. Rev, 2007, 107, 1387-1407; D. Wong, S. J. Lippard, Nature Rev. Drug Disc., 2005, 4, 307-320; S. G. Chaney, S. L. Campbell, E. Bassett, Y. Wu, Crit. Rev. Oncol. Hematol., 2005, 53, 3-11; L. Kelland. Nature Reviews Cancer, 2007, 7, 573-584; J. Reedijk, Eur. J. Inorg. Chem., 2009, 19, 1303-1312; S. V. Zutphen, J. Reedijk, Coord. Chem. Rev., 2005, 249, 2845-2853; S. Ahmad, A. Isab, S. Ali, Transition Met. Chem., 2006, 31, 1003-1016; S. Ahmad, Chemistry & Biodiversity, 2010, 7, 543-566; M. A. Fuertes, J. Castilla, C. Alonso, J. M. Perez. Curr. Med. Chem., 2003, 10, 257; S. Komeda, Metallomics, 2011, 3, 650-655; and S. Dasari, P. B. Tchounwou, Eur. J. Pharmacol., 2014, 740, 364-378, each incorporated herein by reference in their entirety. The clinical use of cisplatin, however, is restricted by the occurrence of serious side effects, such as nephrotoxicity, neurotoxicity and gastrointestinal toxicity. See S. Dasari et al.; J. T. Hartmann, H.-P. Lipp, Expert Opin. Pharmacother., 2003, 4, 889-901; V. M. Piccolini, M. G. Bottone, G. Bottiroli, S. A. De Pascali, F. P. Fanizzi, G. Bemocchi, Cell Biol. Toxicol., 2013, 29, 339-353; and R. P. Miller, R. K. Tadagavadi, G. Ramesh, W. B. Reeves, Toxins, 2010, 2, 2490-2518, each incorporated herein by reference in their entirety. In addition, many tumor cells display inherent or acquired resistance to platinum-based drugs, which further limits their utility. See L. Galluzzi, L. Senovilla, I. Vitale, J. Michels, I. Martins, O. Kepp, M. Castedo, G. Kroemer, Oncogene, 2012, 31, 1869-1883; D. W. Shen, L. M. Pouliot, M. D. Hall, M. M. Gottesman, Pharmacol. Rev., 2012, 64, 706-721; M. Kartalou, J. M. Essigmann, Mutat. Res., 2001, 478, 23-43; and D. J. Stewart, Crit. Rev. Oncol. Hematol., 2007, 63, 12-31, each incorporated herein by reference in their entirety. It is generally accepted that the anticancer effect of cisplatin and its analogues is based on their strong binding to DNA nucleobases, which are the ultimate targets of platinum chemotherapy. See Y. Jung et al.; D. Wong et al.; S. G. Chaney et al.; L. Kelland; J. Reedijk; S. V. Zutphen et al.; S. Ahmad et al; S. Ahmad; M. A. Fuertes et al.; S. Komeda; E. R. Jamieson, S. J. Lippard, Chem. Rev., 1999, 99, 2467-2498; J. Raber, C. Zhu, L. A. Eriksson, J. Phys. Chem. B., 2005, 109, 11006-11015; and M. E. Alberto, V. Butera, N. Russo, Inorg. Chem., 2011, 50, 6965-6971, each incorporated herein by reference in their entirety. Platinum complexes are capable of forming a number of structurally different adducts with DNA, among which the major adduct is bidentate 1,2-intrastrand cross-link, where cis-[Pt(NH₃)₂]²⁺ undergoes cross-linkage between two adjacent guanine N7-atoms. The distortion of double helix as a result of this interaction is recognized by the cellular proteins. The cellular processing of platinated adducts affects the transcription machinery of the cell and eventually leads to cancer cell death. See Y. Jung et al.; D. Wong et al.; S. G. Chaney et al.; L. Kelland; J. Reedijk; S. V. Zutphen et al.; S. Ahmad et al; S. Ahmad; M. A. Fuertes et al.; S. Komeda; E. R. Jamieson et al.; and W. H. Ang, M. Myint, S. J. Lippard, J. Am. Chem. Soc., 2010, 132, 7429-35, each incorporated herein by reference in their entirety.

Although the major cellular target of platinum-based drugs is DNA, the platinum drugs undergo several non-selective reactions with a variety of biomolecules in the cytoplasm, such as methionine, glutathione, RNA and proteins. Glutathione, methionine and other sulfur donor ligands have been found to play a role in the metabolism of cisplatin. See S. V. Zutphen et al.; S. Ahmad et al.; M. Kartalou et al.; D. J. Steward; T. Zimmermann, J. V. Burda, Dalton Trans., 2010, 39, 1295-1301; J. M. Teuben, M. R. Zubiri, J. Reedijk, J. Chem. Soc., Dalton Trans., 2000, 3, 369-372; R. E. Norman, J. D. Ranford, P. J. Sadler, Inorg. Chem., 1992, 31, 877; and L. Messori, A. Merlino, Coord. Chem. Rev., 2016, 315, 67-89, each incorporated herein by reference in their entirety. For example, cis/trans-[Pt(L-methionine)₂]²⁺ is a metabolite detected in the urine of cisplatin-treated patients. See R. E. Norman et al. Platinum compounds are also known to inhibit the activity of thioredoxin reductase, the enzyme that contains selenocysteine at its active site. See Y.-C. Lo, T.-P. Ko, W.-C. Su, T.-L. Su, A. H.-J. Wang, J. Inorg. Biochem., 2009, 103, 1082-1092; and S. Prast-Nielsen, M. Cebula, I. Fader, E. S. J. Amer, Free Radical Biol. & Med., 2010, 49, 1765-1778, each incorporated herein by reference in their entirety. Reactions of cisplatin with L-selenomethionine have been investigated by NMR and mass spectrometry, and the formation of several selenomethionine platinum complexes has been reported. See Q. Liu, J. Lin, P. Jiang, J. Zhang, L. Zhu, Z. Guo, Eur. J. Inorg. Chem., 2002, 2002, 2170; Q. Liu, J. Zhang, X. Ke, Y. Mei, L. Zhu, Z. Guo, J. Chem. Soc., Dalton Trans., 2001, 101, 911; and K. M. Williams, R. P. Dudgeon, S. C. Chmely, S. R. Robey, Inorg. Chien. Acta, 2011, 368, 187-193, each incorporated herein by reference in their entirety. A number of cis-amine platinum complexes containing selenolates and selenoether ligands have been synthesized and some of them were evaluated for their cytotoxicity. See S. M. Chopade, P. P. Phadnis, A. S. Hodage, A. Wadawale, V. K. Jain, Inorg. Chim. Acta, 2015, 427, 72-80; S. M. Chopade, P. P. Phadnis, A. Wadawale, A. S. Hodage, V. K. Jain. Inorg. Chim. Acta, 2012, 385, 185-189; M. Carland, B. F. Abrahams, T. Rede, J. Stephenson, V. Murray, W. A. Denny, W. D. McFadyen, Inorg. Chim. Acta, 2006, 359, 3252-3256; C. Rothenburger, M. Galanski, V. B. Arion, H. Görls, W. Weigand, B. K. Keppler, Eur. J. Inorg. Chem., 2006, 2006, 3746-3752; and A. L. Fuller, F. R. Knight, A. M. Z. Slawin, J. D. Woollins, Eur. J. Inorg. Chem., 2010, 2010, 4034-4043, each incorporated herein by reference in their entirety. However, the reports on the platinum complexes of selenones are very much limited, although the complexes of thiones have been studied extensively. See P. J. Hendra, Z. Jovic, Spectrochim. Acta. A, 1968, 24, 1713-1720; D. Fregona, R. Graziani, G. Faraglia, U. Caselato, S. Sitran, Polyhedron, 1996, 15, 2523-2533; J. Moussa, K. M.-C. Wong, X. F. Le Goff, M. N. Rager, C. K.-M. Chan, V. W.-W. Yam, H. Amouri, Organometallics, 2013, 32, 4985-4992; M. M. Kubicki, T. Glowiak, Mater. Sci. III (Poland), 1977, 1-2, 35-38; A. Zainelabdeen A. Mustafa, M. Monim-ul-Mehboob, M. Y. Jomaa, M. Altaf, M. Fettouhi, A. A. Isab, M. I. M. Wazeer, H. Stoeckli-Evans, G. Bhatia, V. Dhuna, J. Coord. Chem., 2015, 68, 3511-3524; A Zainelabdeen A. Mustafa, M. Altaf, M. Monim-ul-Mehboob, M. Fettouhi, M. I. M. Wazeer, A. A. Isab, V. Dhuna, G. Bhatia, K. Dhuna, Inorg. Chem. Comm., 2014, 44, 159-163; H. Sadaf, A. A. Isab, S. Ahmad, A. Espinosa, M. Mas-Montoya, I. U. Khan, Ejaz, S. Rehman, M. A. J. Ali, M. Saleem, J. Ruiz, C. Janiak, J. Mol. Struc., 2015, 1085, 155-161; Seerat-ur-Rehman, A. A. Isab, M. N. Tahir, T. Khalid, M. Saleem, H. Sadaf, S. Ahmad, Inorg. Chem. Comm., 2013, 36, 68-71; J. Lin, G. Lu, L. M. Daniels, X. Wei, J. B. Sapp, Y. Deng, J. Coord. Chem., 2008, 61, 2457-2469; J. Calvo, J. S Cases, E. Garcia-Martinez, Y. Parajo, A. Sanchez-Gonzalez, J. Sordo, Z. Anorg. Alleg. Chem., 2004, 630, 215-216; J. Jolley, W. I. Cross, R. G. Pritchard, C. A. McAuliffe, K. B. Nolan, Inorg. Chim. Acta, 2001, 315, 36-43; M. Mizota, Y. Yokoyama, K. Sakai, Acta Cryst. E, 2005, 61, m1433-m1435; S. Wang, R. J. Staples, J. P. Fackler Jr, Acta Cyst. C, 1994, 50, 889-891; and D. M. L. Goodgame, R. W. Rollins, A. M. Z. Slawin, D. J. Williams, P. W. Zard, Inorg. Chum. Acta, 1986, 120, 91-101, each incorporated herein by reference in their entirety. The structural studies on thione complexes reveal a square planar geometry around platinum(II) and thione ligands coordination either in monodentate through sulfur atom or in bidentate S,N-chelating modes. See A. Zainelabdeen A. Mustafa et al.; A. Zainelabdeen A. Mustafa et al; H. Sadaf et al.; Seerat-ur-Rehman et all; J. Lin et al.; J. Calvo et al; J. Jolley et al; M. Mizota et al; S. Wang et al.; and D. M. L. Goodgame et al. The complexes are usually stabilized by extensive hydrogen bonding interactions. The structural and anticancer studies of these complexes have seen interest. See A. Zainelabdeen A. Mustafa, M. Monim-ul-Mehboob, M. Y. Jomaa, M. Altaf, M. Fettouhi, A. A. Isab, M. I. M. Wazeer, H. Stoeckli-Evans, G. Bhatia, V. Dhuna, J. Coord. Chem., 2015, 68, 3511-3524; A. Zainelabdeen A. Mustafa, M. Altaf, M. Monim-ul-Mehboob, M. Fettouhi, M. I. M. Wazeer, A. A. Isab, V. Dhuna, G. Bhatia, K. Dhuna, Inorg. Chem. Comm., 2014, 44, 159-163; H. Sadaf et al; and Seerat-ur-Rehman et al. Keeping in mind the chemotherapeutic effects of organoselenium compounds, the structural features and cytotoxicity of platinum(II) complexes of selenones is of interest. With this objective, herein is disclosed the synthesis, spectral as well as structural characterization and evaluation of anticancer properties of some platinum(II) complexes of selenone ligands. The X-ray structures of the complexes as well as the ⁷⁷Se and ¹⁹⁵Pt NMR chemical shifts provide valuable information regarding platinum(II) complexes of selenones. The structures of the selenones used in this study and their resonance assignments are given in Scheme 1.

The inventors demonstrate herein the synthesis of these complexes and disclose their spectral and structural features as well as their anticancer properties.

### BRIEF SUMMARY OF THE INVENTION

The following provides a summary of certain exemplary embodiments of the present invention. This summary is not an extensive overview and is not intended to identify key or critical aspects or elements of the present invention or to delineate its scope.

The invention is directed to platinum(II) complexes with selenones and to methods of treating cancer using these complexes. Several classes and seven complexes are exemplified; e.g., Complexes 1, 2, 3, 4, 5, 6, and 7. The invention includes these complexes as well as their structural variants, for example, complexes having additional non-hydrogen ring substituents or anions other than chloride.

### BRIEF DESCRIPTION OF THE DRAWINGS

The foregoing summary, as well as the following detailed description of preferred embodiments of the invention, will be better understood when read in conjunction with the appended drawings. It should be understood, however, that the invention is not limited to the precise features, arrangements, or instrumentalities shown.

FIG. 1. A view of the molecular structure of compound 3, with the atom labelling. The displacement ellipsoids are drawn at the 50% probability level. The unlabeled atoms are related to the labelled atoms by symmetry code: −x+1, −y+1, −z+1.

FIG. 2. A view of the molecular structure of complex 5, with the atom labelling. The displacement ellipsoids are drawn at the 50% probability level.

FIGS. 3A, 3B and 3C. The mass spectra of methanol solution of complex 4 (FIG. 3A), the solution of complex 4 in methanol:water (1:1, v/v) (FIG. 3B), and the interacting system containing complex 4 (10 μM) and the physiological levels of L-cysteine (290 μM) and reduced glutathione (6 μM), measured 24 h after preparation (FIG. 3C). The identified ionic species are noted.

FIG. 4. The ¹⁹⁵PtNMR spectrum of complex 3.

FIG. 5. The ¹⁹⁵Pt NMR spectrum of complex 7.

FIG. 6. The crystal packing of compound 3, viewed along the a axis. The N—H . . . Cl, C—H . . . Cl and C—H . . . Se hydrogen bonds (dashed lines) lead to the formation of a three-dimensional supramolecular structure.

FIG. 7. The crystal packing of complex 5, observed along the a axis. The N—H . . . Cl and C—H . . . Cl hydrogen bonds (dashed lines).

FIG. 8. Chemical structures of the selenones forming part of the core structures of complexes (1), (2)-(5), (6) and (7), described by formulas HL1, HL2-HL5, HL6, and HL7 respectively.

### DETAILED DESCRIPTION OF THE INVENTION

The invention is directed to platinum(II) complexes with selenone ligands including complexes (1), (2), (3), (4), (5), (6), and (7), the core structures of these complexes, chemical variants of these core structures, the complexes with various counterions or sulfur-containing compounds, and to methods that induce cytotoxicity in cancer or tumor cells.

Platinum(II) complexes (1)-(7) with selenones (HLn), having the general formulae [Pt(HLn)₄]Cl₂, were prepared and characterized by elemental analyses, IR and NMR (¹H, ¹³C, ⁷⁷Se&¹⁹⁵Pt) methods and two of them, [Pt(N-ethylimidazolidine-2-selenone)₄]Cl₂ (3) and [Pt(N-isopropylimidazolidine-2-selenone)₄]Cl₂ (5) by X-ray crystallography. A decrease in the IR frequency of the >C═Se mode and an upfield shift in ¹³C NMR for the >C═Se resonance of selenones was found to be consistent with the selenium coordination to platinum(II). The compounds 3 and 5 consist of [Pt(HL)₄]²⁺ complex ions and chloride counter ions. The platinum(II) atoms in both cations adopted a distorted square planar geometry. Interaction studies with sulfur-containing biomolecules revealed their ability to form a variety of coordination and recombination intermediates as well as oxidized species with L-cysteine and reduced glutathione.

The in vitro antitumor activity of the complexes, as well as cisplatin, were evaluated by MTT assay against human ovarian carcinoma A2780 and its cisplatin-resistant subline A2780R, against human prostate cancer cell line 22Rv,1 and against the human breast adenocarcinoma MCF-7 cell line. The results indicated that two of the complexes, involving the N-propylimidazolidine-2-selenone ligand, namely (4) [Pt(HL4)₄]Cl₂ and (5) [Pt(HL5)₄]Cl₂, were effective against the A2780 cells (IC₅₀=30.8 μM and 44.7 μM respectively). This degree of efficacy is comparable to that of cisplatin (IC₅₀=26.8 μM).

The present disclosure will be better understood with reference to the following definitions:

As used herein “compound” and “complex” are used interchangeably and are intended to refer to a chemical entity, whether in a solid, liquid or gaseous phase and whether in a crude mixture or a purified and isolated form. The platinum(II) complexes of the invention may be referred to as 1, complex 1, or compound 1; 2, complex 2 or compound 2; 3, complex 3 or compound 3; 4, complex 4 or compound 4; 5, complex 5 or compound 5; 6, complex 6 or compound 6; or 7, complex 7 or compound 7.

Platinum(II) or Pt(II) describes platinum in an oxidation state of +2. One example of a platinum(II) compound is platinum chloride having the chemical formula PtCl₂.

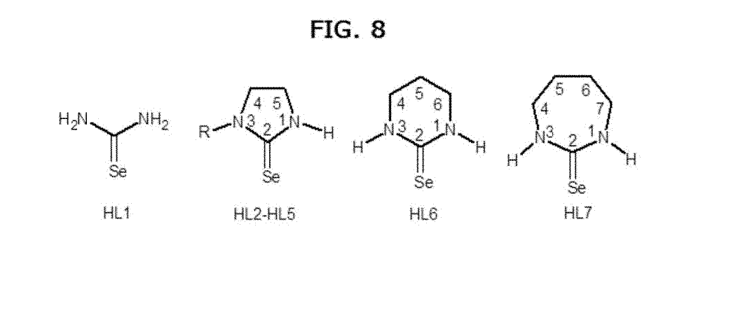

Selenones. The term “selenone” or “selenone ligand” as used herein describes molecules containing a selenourea type structure where Se is double-bonded to a carbon atom and the carbon atom is bound to two nitrogen atoms. A selenone as described herein can have a ring structure or an open structure with >C═Se moiety. Examples of selenones or selenone ligands according to the invention are shown in FIG. 8. When these selenones form complexes with Pt, they may do so by forming a bond between Se and Pt as shown in the figures. The structural formulae of some selenone ligands used to produce Pt(II) selenone complexes shown herein is: HL1, selenourea; HL2, R=H, imidazolidine-2-selenone; HL3, R=C₂H₅, N-ethylimidazolidine-2-selenone; HL4, R=C₃H₂, N-propylimidazolidine-2-selenone; HL5, R=i-C₃H₇, N-isopropylimidazolidine-2-selenone; HL6, 1,3-diazinane-2-selenone; HL7, 1,3-diazepane-2-selenone. The numbering to the Pt(II)-selenone complexes has been assigned as follows: [Pt(HL1)₄]Cl₂=1, [Pt(HL2)₄]Cl₂=2, [Pt(HL3)₄]Cl₂=3, [Pt(HL4)₄]Cl₂=4, [Pt(HL5)₄]Cl₂=5, [Pt(HL6)₄]Cl₂=6 and [Pt(HL7)₄]Cl₂=7. Selenone ligands react with K₂PtCl₄ to form complexes of the type [Pt(HL₄)]Cl₂ in which the ligands exist in the selenone form both in solution as well as in the solid state.

The terms “anion” or “counter-anion” refer to an anion, preferably a pharmaceutically acceptable anion that is associated with a positively charged platinum(II) complex core. Non-limiting examples of pharmaceutically acceptable counter-anions include halides such as fluoride, chloride, bromide, iodide; nitrate; sulfate; phosphate; amide; methanesulfonate; ethanesulfonate; p-toluenesulfonate, salicylate, malate, maleate, succinate, tartrate; citrate; acetate; perchlorate; trifluoromethanesulfonate (triflate); acetylacetonate; hexafluorophosphate; and hexafluoroacetylacetonate. In some embodiments, a complex of the invention may be further contacted, coordinated or combined with a sulfur-containing molecule, such as glutathione, cysteine, 2-ME, or DTT.

Variants. Complexes that vary from those described by complexes (1)-(7) depicted above may comprise Pt(II) complexes comprising the same or different selenones, complexes comprising selenones other than HL1-HL7 (FIG. 8), such as further substituted variants of HL1-HL7, complexes with different counterions, such as those with one or two non-chloride anions, complexes with different degrees of hydration, or complexes further combined with other molecules such as sulfur-containing molecules or modified or further substituted selenones. A selenone component of the complex may be further substituted, for example, it may have one or more non-hydrogen substituents on the core or ring structures depicted for selenones HL1-HL7 in FIG. 8. The points of substitution include one or both of the nitrogen atoms depicted by selenone HL1; one or both of the ring nitrogen atoms depicted by selenones HL2-HL5, at positions 4 and 5 of HL2-HL5, one or both of the ring nitrogen atoms depicted in selenone HL6, on positions 4, 5 and 6 of HL6, one or both of the ring nitrogens of selenone HL7 or at positions 4, 5, 6, or 7 of HL7. Heterocyclic selenones are stable ligands and can be substituted often without substantial effects on a Pt(II)selenone complex's cytotoxic and anti-cancer properties.

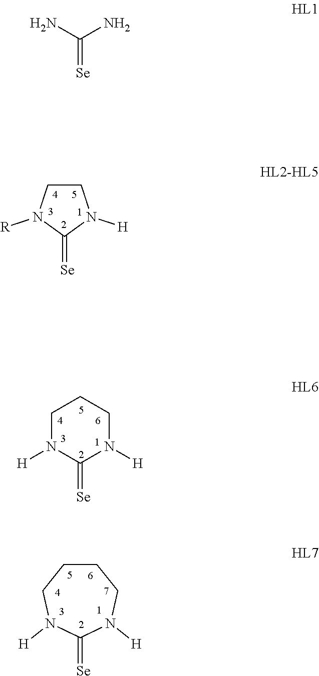

Selenone ligands: selenourea (HL1), imidazolidine-2-selenone (HL2), N-ethylimidazolidine-2-selenone (HL3), N-propylimidazolidine-2-selenone (HL4), N-isopropylimidazolidine-2-selenone (HL5), diazinane-2-selenone (HL6), and diazepane-2-selenone (HL7). Organic molecules with >C═Se are commonly called selenones.

Other substituents that may appear on the core selenone structures of complexes (1)-(7) or on the structures shown in FIG. 8 include, but are not limited those defined below.

The term alkyl, as used herein, unless otherwise specified, refers to a straight or branched hydrocarbon fragment such as a C₁-C₆ group. Non-limiting examples of such hydrocarbon fragments include methyl, ethyl, propyl, isopropyl, butyl, isobutyl, t-butyl, pentyl, isopentyl, neopentyl, hexyl, isohexyl, 3-methylpentyl, 2,2-dimethylbutyl, and 2,3-dimethylbutyl. As used herein, the term “cycloalkyl” refers to a cyclized alkyl group. Exemplary cycloalkyl groups include, but are not limited to, cyclopropyl, cyclobutyl, cyclopentyl, cyclohexyl, norbornyl, and adamantyl. Branched cycloalkyl groups, for example, 1-methylcyclopropyl and 2-methylcyclopropyl groups, are included in the definition of cycloalkyl as used in the present disclosure. The term “alkenyl” refers to a straight, branched, or cyclic hydrocarbon fragment containing at least one C═C double bond. Exemplary alkenyl groups include, without limitation, 1-propenyl, 2-propenyl (or “allyl”), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, and 9-decenyl. The term “aryl”, as used herein, and unless otherwise specified, refers to phenyl, biphenyl, naphthyl, anthracenyl, and the like. The term “heteroaryl” refers to an aryl group where at least one carbon atom is replaced with a heteroatom (e.g. nitrogen, oxygen, sulfur) and can be indolyl, furyl, imidazolyl, triazolyl, triazinyl, oxazolyl, isoxazolyl, thiazolyl, isothiazolyl, pyrazolyl, pyrrolyl, pyrazinyl, tetrazolyl, pyridyl (or its N-oxide), thienyl, pyrimidinyl (or its N-oxide), IH-indolyl, isoquinolyl (or its N-oxide), or quinolyl (or its N-oxide), for example. As used herein, the term “substituted” refers to at least one hydrogen atom that is replaced with a non-hydrogen group, provided that normal valencies are maintained and that the substitution results in a stable compound. When a compound or a R group is noted as “optionally substituted”, the substituents are selected from the exemplary group including, but not limited to, aroyl (as defined hereinafter); halogen (e.g. chlorine, bromine, fluorine or iodine); alkoxy (i.e. straight or branched chain alkoxy having 1 to 10 carbon atoms, and includes, for example, methoxy, ethoxy, propoxy, isopropoxy, butoxy, isobutoxy, sec-butoxy, tert-butoxy, pentoxy, isopentoxy, hexyloxy, heptyloxy, octyloxy, nonyloxy, and decyloxy); cycloalkyloxy including cyclopentyloxy, cyclohexyloxy, and cycloheptyloxy; aryloxy including phenoxy and phenoxy substituted with halogen, alkyl, alkoxy, and haloalkyl (which refers to straight or branched chain alkyl having 1 to 8 carbon atoms which are substituted by at least one halogen, and includes, for example, chloromethyl, bromomethyl, fluoromethyl, iodomethyl, 2-chloroethyl, 2-bromoethyl, 2-fluoroethyl, 3-chloropropyl, 3-bromopropyl, 3-fluoropropyl, 4-chlorobutyl, 4-fluorobutyl, dichloromethyl, dibromomethyl, difluoromethyl, diiodomethyl, 2,2-dichloroethyl, 2,2-dibromoethyl, 2,2-difluoroethyl, 3,3-dichloropropyl, 3,3-difluoropropyl, 4,4-dichlorobutyl, 4,4-difluorobutyl, trichloromethyl, trifluoromethyl, 2,2,2-tri-fluoroethyl, 2,3,3-trifluoropropyl, 1,1,2,2-tetrafluoroethyl, 2,2,3,3-tetrafluoropropyl); hydrocarbyl; arylalkyl; hydroxy; alkoxy; oxo; alkanoyl; alkanoyloxy; amino; alkylamino; arylamino; arylalkylamino; disubstituted amines (e.g., in which the two amino substituents are selected from a group including, but not limited to, alkyl, aryl, or arylalkyl); alkanoylamino; thiol; alkylthio; arylthio; arylalkylthio; alkylthiono; arylthiono; aryalkylthiono; alkylsulfonyl; arylsulfonyl; arylalkylsulfonyl; sulfonamido (e.g., —SO₂NH₂); substituted sulfonamide; nitro; cyano; carboxy; carbamyl (e.g., —CONH₂, —CONHalkyl, —CONHaryl, —CONHarylalkyl or cases where there are two substituents on one nitrogen from alkyl, aryl, or arylalkyl); alkoxycarbonyl; aryl; heteroarylcarbonyl; heterocyclyl; and mixtures thereof and the like. The substituents may be either unprotected, or protected as necessary, as known to those skilled in the art, for example, as taught in Greene, et al., “Protective Groups in Organic Synthesis”, John Wiley and Sons, Second Edition, 1991, hereby incorporated by reference in its entirety). The term “heterocyclyl” as used in this disclosure refers to a 3-8, preferably 4-8, more preferably 4-7 membered monocyclic ring or a fused 8-12 membered bicyclic ring which may be saturated or partially unsaturated, which monocyclic or bicyclic ring contains 1 to 4 heteroatoms selected from oxygen, nitrogen, silicon, or sulfur. Examples of such monocyclic rings include oxaziridinyl, homopiperazinyl, oxiranyl, dioxiranyl, aziridinyl, pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydropyranyl, tetrahydrofuranyl, dihydropyranyl, tetrahydropyranyl, tetrahydropyridyl, tetrahydropyrimidinyl, tetrahydrothiophenyl, tetrahydrothiopyranyl, diazepanyl, and azepanyl. Examples of such bicyclic rings include indolinyl, isoindolinyl, benzopyranyl, quinuclidinyl, 2,3,4,5-tetrahydro-1,3,benzazepine, 4-(benzo-1,3,dioxol-5-methyl)piperazine, and tetrahydroisoquinolinyl. Further, “substituted heterocyclyl” may refer to a heterocyclyl ring which has additional (e.g. one or more) oxygen atoms bonded to the ring atoms of parent heterocycyl ring. An example of a heterocyclyl substituted with one or more oxygen atoms is 1,1-dioxido-1,3-thiazolidinyl. The term “alkylthio” as used in this disclosure refers to a divalent sulfur with alkyl occupying one of the valencies and includes the groups methylthio, ethylthio, propylthio, butylthio, pentylthio, hexylthio, and octylthio. The term “alkanoyl” as used in this disclosure refers to an alkyl group having 2 to 18 carbon atoms that is bound with a double bond to an oxygen atom. Examples of alkanoyl include, acetyl, propionyl, butyryl, isobutyryl, pivaloyl, valeryl, hexanoyl, octanoyl, lauroyl, and stearoyl. Examples of aroyl are benzoyl and naphthoyl, and “substituted aroyl” may refer to benzoyl or naphthoyl substituted by at least one substituent including those selected from halogen, amino, nitro, hydroxy, alkyl, alkoxy and haloalkyl on the benzene or naphthalene ring. The term “arylalkyl” as used in this disclosure refers to a straight or branched chain alkyl moiety having 1 to 8 carbon atoms that is substituted by an aryl group or a substituted aryl group having 6 to 12 carbon atoms, and includes benzyl, 2-phenethyl, 2-methylbenzyl, 3-methylbenzyl, 4-methylbenzyl, 2,4-dimethylbenzyl, 2-(4-ethylphenyl)ethyl, 3-(3-propylphenyl)propyl. The term “heteroarylcarbonyl” as used in this disclosure refers to a heteroaryl moiety with 5 to 10 membered mono- or fused-heteroaromatic ring having at least one heteroatom selected from nitrogen, oxygen and sulfur as mentioned above, and includes, for example, furoyl, nicotinoyl, isonicotinoyl, pyrazolylcarbonyl, imidazolylcarbonyl, pyrimidinylcarbonyl, and benzimidazolyl-carbonyl. Further, “substituted heteroarylcarbonyl” may refer to the above mentioned heteroarylcarbonyl which is substituted by at least one substituent selected from halogen, amino, vitro, hydroxy, alkoxy and haloalkyl on the heteroaryl nucleus, and includes, for example, 2-oxo-1,3-dioxolan-4-ylmethyl, 2-oxo-1,3-dioxan-5-yl. “Vinyl” refers to an unsaturated substituent having at least one unsaturated double bond and having the formula CH2=CH—. Accordingly, said “substituted vinyl” may refer to the above vinyl substituent having at least one of the protons on the terminal carbon atom replaced with alkyl, cycloalkyl, cycloalkylalkyl, aryl, substituted aryl, heteroaryl or substituted heteroaryl. The term “hydrocarbyl” as used herein refers to a univalent hydrocarbon group containing up to about 24 carbon atoms (i.e. a group containing only carbon and hydrogen atoms) and that is devoid of olefinic and acetylenic unsaturation, and includes alkyl, cycloalkyl, alkyl-substituted cycloalkyl, cycloalkyl-substituted cycloalkyl, cycloalkylalkyl, aryl, alkyl-substituted aryl, cycloalkyl-substituted aryl, arylalkyl, alkyl-substituted aralkyl, and cycloalkyl-substituted aralkyl. Further, functionally-substituted hydrocarbyl groups may refer to a hydrocarbyl group that is substituted by one or more functional groups selected from halogen atoms, amino, nitro, hydroxy, hydrocarbyloxy (including alkoxy, cycloalkyloxy, and aryloxy), hydrocarbylthio (including alkylthio, cycloalkylthio, and arylthio), heteroaryl, substituted heteroaryl, alkanoyl, aroyl, substituted aroyl, heteroarylcarbonyl, and substituted heteroarylcarbonyl. In some embodiments, hydrogen is replaced by C1-C6 alkyl on atoms not participating in the Pt(II)-Se bond. The hydrophobicity or hydrophilicity of the complex may be adjusted by selecting appropriate substituents for the selenone component of the complex or by selection of different counteranions or complexing components. A size and relative degree of hydrophilicity or hydrophobicity suitable for a particular mode of administration and uptake of the complex at a desired site of action. For example, a complex may be made more hydrophobic by substitution of the selenone moiety with alkyl or aryl to increase its ability to cross a lipid bilayer or to interact with non-polar compounds. Alternatively it may be made more hydrophilic by substitution of the selenone moiety with a more polar substituent to facilitate serum binding, adsorption into water-containing bodily fluids, or interaction with polar compounds.

Compositions. In many embodiments, the platinum(II) complexes of the invention, the salt thereof, the solvate thereof, a prodrug thereof, or a combination thereof is formulated as a pharmaceutically acceptable composition. As used herein, a “composition” refers to a mixture of the active ingredient with at least one other chemical component, such as a pharmaceutically acceptable carrier or excipient. One purpose of a composition is to facilitate administration of the platinum(II) complex of the invention, the salt thereof, the solvate thereof, the prodrug thereof, or a combination thereof to a subject. Depending on the intended mode of administration (oral, parenteral, or topical), the composition can be in the form of solid, semi-solid, liquid, or aerosol dosage forms, such as tablets, suppositories, pills, capsules, powders, liquids, or suspensions, preferably in unit dosage form suitable for single administration of a precise dosage. The phrase “pharmaceutically acceptable” as used herein refers to compounds, counterions, materials, compositions, and/or dosage forms which are, within the scope of sound medical judgment, suitable for use in contact with the tissues of human beings and animals without excessive toxicity, irritation, allergic response, or other problem or complication and commensurate with a reasonable benefit/risk ratio. Therefore, the composition refers to the combination of an active ingredient with a carrier or excipient, inert or active, making the composition especially suitable for diagnostic or therapeutic use in vivo, ex vivo, or in vitro.

As used herein, a pharmaceutically acceptable carrier refers to a carrier or diluent that does not cause significant irritation to an organism, does not abrogate the biological activity and properties of the administered active ingredient, and/or does not interact in a deleterious manner with the other components of the composition in which it is contained. The term “carrier” encompasses any excipient, binder, diluent, filler, salt, buffer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS® (BASF; Florham Park, N.J.). An “excipient” refers to an inert substance added to a composition to further facilitate administration of a compound. Examples, without limitation, of excipients include calcium carbonate, calcium phosphate, various sugars and types of starch, cellulose derivatives, gelatin, vegetable oils, and polyethylene glycols.

In other embodiments, the composition has various release rates (e.g. controlled release or immediate release). Immediate release refers to the release of an active ingredient substantially immediately upon administration. In another embodiment, immediate release occurs when there is dissolution of an active ingredient within 1-20 minutes after administration. Dissolution can be of all or less than all (e.g., about 70%, about 75%, about 80%, about 85%, about 90%, about 91%, about 92%, about 93%, about 94%, about 95%, about 96%, about 97%, about 98%, about 99%, about 99.5%, 99.9%, or 99.99%) of the active ingredient. In another embodiment, immediate release results in complete or less than complete dissolution within about 1 hour following administration. Dissolution can be in a subject's stomach and/or intestine. In one embodiment, immediate release results in dissolution of an active ingredient within 1-20 minutes after entering the stomach. For example, dissolution of 100% of an active ingredient can occur in the prescribed time. In another embodiment, immediate release results in complete or less than complete dissolution within about 1 hour following rectal administration. In some embodiments, immediate release is through inhalation, such that dissolution occurs in a subject's lungs.

Controlled-release or sustained-release refers to the release of an active ingredient from a composition or dosage form in which the active ingredient is released over an extended period of time. In one embodiment, controlled-release results in dissolution of an active ingredient within 20-180 minutes after entering the stomach. In another embodiment, controlled-release occurs when there is dissolution of an active ingredient within 20-180 minutes after being swallowed. In another embodiment, controlled-release occurs when there is dissolution of an active ingredient within 20-180 minutes after entering the intestine. In another embodiment, controlled-release results in substantially complete dissolution after at least 1 hour following administration. In another embodiment, controlled-release results in substantially complete dissolution after at least 1 hour following oral administration. In another embodiment, controlled-release results in substantially complete dissolution after at least 1 hour following rectal administration. In another embodiment, controlled-release results in substantially complete release of the active component after or over at least 1, 2, 4, 8, 12, 24 hours or 2, 3, 4, 5, 6, or 7 days (or any intermediate value within this range) following administration including a depot administration into or around a tumor. In one embodiment, the composition is not a controlled-release composition.

Solid dosage forms for oral administration can include capsules, tablets, pills, powders, and granules. In such solid dosage forms, the active ingredient is ordinarily combined with one or more adjuvants appropriate to the indicated route of administration. If administered per os, the active ingredient can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering ingredients such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting ingredients, emulsifying and suspending ingredients, and sweetening, flavoring, and perfuming ingredients.

For therapeutic purposes, formulations for parenteral administration can be in the form of aqueous or non-aqueous isotonic sterile injection solutions or suspensions. The term “parenteral”, as used herein, includes intravenous, intravesical, intraperitoneal, subcutaneous, intramuscular, intralesional, intracranial, intrapulmonal, intracardial, intrastemal, and sublingual injections, or infusion techniques. These solutions and suspensions can be prepared from sterile powders or granules having one or more of the carriers or diluents mentioned for use in the formulations for oral administration. The active ingredient can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations, for example, sterile injectable aqueous or oleaginous suspensions can be formulated according to the known art using suitable dispersing or wetting ingredients and suspending ingredients. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids, such as oleic acid, find use in the preparation of injectables. Dimethyl acetamide, surfactants including ionic and non-ionic detergents, polyethylene glycols can be used. Mixtures of solvents and wetting ingredients such as those discussed above are also useful.

Suppositories for rectal administration can be prepared by mixing the active ingredient with a suitable non-irritating excipient, such as cocoa butter, synthetic mono-, di-, or triglycerides, fatty acids, and polyethylene glycols that are solid at ordinary temperatures but liquid at the rectal temperature and will therefore melt in the rectum and release the drug.

Topical administration can also involve the use of transdermal administration such as transdermal patches or iontophoresis devices. Formulation of drugs is discussed in, for example, Hoover, John E., Remington's Pharmaceutical Sciences, Mack Publishing Co., Easton, Pa., 1975. Another example of includes Liberman, H. A. and Lachman, L., Eds., Pharmaceutical Dosage Forms, Marcel Decker, New York, N.Y., 1980, which is incorporated herein by reference in its entirety).

Other active ingredients. In some embodiments, other active ingredients, in addition to the platinum(II)complex may be incorporated into a composition or separately administered in conjunction with a platinum(II) complex. In one embodiment, the composition is used for treating cancer and further comprises a second active ingredient, such as a chemotherapeutic or immunotherapeutic agent, for the treatment or prevention of neoplasm, of tumor or cancer cell division, growth, proliferation and/or metastasis in the subject; induction of death or apoptosis of tumor and/or cancer cells; and/or any other form of proliferative disorder. Exemplary chemotherapeutic agents include, without limitation, aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosfamide, irinotecan, lomustine, mechlorethamine, melphalan, mercaptopurine, methotrexate, mitomycin, mitoxantrone, pentostatin, procarbazine, topotecan, vinblastine, vincristine, retinoic acid, oxaliplatin, carboplatin, 5-fluorouracil, teniposide, amasacrine, docetaxel, paclitaxel, vinorelbine, bortezomib, clofarabine, capecitabine, actinomycin D, epirubicin, vindesine, methotrexate, 6-thioguanine, tipifamib, imatinib, erlotinib, sorafenib, sunitinib, dasatinib, nilotinib, lapatinib, gefitinib, temsirolimus, everolimus, rapamycin, bosutinib, pzopanib, axitinib, neratinib, vatalanib, pazopanib, midostaurin, enzastaurin, trastuzumab, cetuximab, panitumumab, rituximab, bevacizumab, mapatumumab, conatumumab, and lexatumumab. The composition may comprise 0.1-50 wt % of the second active ingredient, preferably 10-40 wt %, more preferably 10-20 wt %, relative to the weight of the first active ingredient.

Subjects. The terms “patient”, “subject”, and “individual” are used interchangeably. As used herein, they refer to individuals suffering from a disease, at risk of further progression of a disease, or at risk of acquiring or developing the disease. None of these terms require that the individual be under the care and/or supervision of a medical professional.

These terms generally refer to humans, but also apply to mammals, avians and other animals, especially domesticated or ecologically or commercially valuable animals. Mammals include non-human primates, such as chimpanzees, and other apes and monkey species, farm animals, such as cattle, horses, sheep, goats, swine, domestic animals, such as rabbits, dogs, and cats, laboratory animals including rodents, such as rats, mice and guinea pigs, and the like. In a preferred embodiment, the subject is a human.

A subject in need of treatment includes a subject already with a disease such as cancer, a subject who does not yet experience or exhibit symptoms of the disease, and a subject who is predisposed to the disease for example based on family history or genetic profile. In preferred embodiments, the subject is a person who is predisposed to cancer such as a person with a family history of cancer. In another embodiment, the subject refers to a cancer patient who has been previously administered/treated with cisplatin and have cisplatin resistance, for example in the form of high ERCC1 mRNA levels, overexpression of HER-2/neu, activation of the PI3-K/Akt pathway, loss of p53 function, and/or overexpression of antiapoptotic bcl-2).

The term active ingredient, as used herein, refers to an ingredient in the composition that is biologically active, for example, the platinum(II) complexes disclosed herein, a salt thereof, a prodrug thereof, or a solvate thereof. Other active ingredients include, but are not limited to, those that exert a substantial pharmacokinetic or pharmacodynamic activity when in admixture with a platinum(II) complex, for example, other anti-cancer drugs, immunopotentiators, or other agents.

Antitumor properties may be evaluated by methods known in the art, including these described by and incorporated by reference to Y. F. To, R. W.-Y. Sun, Y. Chen, V. S.-F. Chan, W.-Y. Yu, P. K.-H. Tam, C.-M. Che and C.-L. S. Lin, Int. J. Cancer, 2009, 124, 1971-1979; C. T. Lum, Z. F. Yang, H. Y. Li, R. W.-Y. Sun, S. T. Fan, R. T. P. Poon, M. C. M. Lin, C.-M. Che and H. F. Kung, hit. J. Cancer, 2006, 118, 1527-1538; C. T. Lum, A. S.-T. Wong, M. C. M. Lin, C.-M. Che and R. W.-Y. Sun, Chem. Commun., 2013, 49, 4364-4366; C.-M. Che, R. W.-Y. Sun, W.-Y. Yu, C.-B. Ko, N. Zhu and H. Sun, Chem. Common., 2003, 1718-1719; Y. Wang, Q.-Y. He, R. W.-Y. Sun, C.-M. Che and J.-F. Chiu, Eur. J. Pharmacol., 2007, 554, 113-122—each incorporated by reference.

Cytotoxic activity. In one embodiment, the IC₅₀ of the platinum(II) complexes is in a range of 0.01-200 μM, 0.1-100 μM, 1-100 μM, 10-90 μM, 20-80 μM, 30-80 μM, 40-80 μM, 50-80 μM, or 50-75 μM. These ranges include all intermediate subranges and values.

As used herein, the term “IC₅₀” refers to a concentration of a platinum(II) complex, the salt thereof, the prodrug thereof, or the solvate thereof, which causes the death of 50% of cancer or proliferating cells in 72 hours (3 days) such as the MCF-7, A2780, A2780R, or 22Rv1 cancer cell lines described herein. The IC₅₀ can be determined by standard cell viability assays, such as, without limitation, ATP test, calcein AM assay, clonogenic assay, ethidium homodimer assay, Evans blue assay, Fluorescein diacetate hydrolysis/propidium iodide staining assay, flow cytometry assay, formazan-based assays (MIT, XTT), green fluorescent protein assay, lactate dehydrogenase assay, methyl violet assay, propidium iodide assay, Resazurin assay, Trypan Blue assay and TUNEL assay. Preferably, a MTT assay and/or a Trypan Blue assay is used.

Biomarkers. Alternatively to use of IC₅₀ values, efficacy of treatment with a platinum(II) complex of the invention may be determined by measuring or detecting a change in one or cancer biomarkers, for example, comparing quantity of biomarkers in a blood or tissue sample before and after a treatment.

A treatment may significantly decrease the concentration of a particular biomarker, for example, by 5, 10, 15, 20, 25, 30, 35, 40, 45, 50, 55, 60, 65, 70, 75, 80, 85, 90, or 100%, compared to a control or pre-treatment value. As used herein, the term “biomarker” refers to a characteristic that is objectively measured and evaluated as an indicator of normal biological processes, pathogenic processes or pharmacological responses to a therapeutic intervention. Biomarkers include ER/PR, HER-2/neu for breast cancer, EGFR, KRAS, UGT1A1 for colorectal cancer, EML4/ALK, EGFR, and KRAS for lung cancer as well as other biomarkers described and incorporated by reference to https://_en.wikipedia.org/wiki/Cancer_biomarkers (last accessed Oct. 5, 2017). Cancer biomarkers are useful in determining the aggressiveness of an identified cancer as well as its likelihood of responding to the treatment. Examples of such prognostic biomarkers include, without limitation, CA125, β2-microglobulin, and EBV DNA. A change or mutation in a biomarker may be detected with a polymerase chain reaction (PCR) assay, DNA microarray, multiplex ligation-dependent probe amplification. (MLPA), single strand conformational polymorphism (SSCP), denaturing gradient gel electrophoresis (DGGE), heteroduplex analysis, and restriction fragment length polymorphism (RFLP). The procedures to detect the mutation are well-known to those of ordinary skill in the art. The concentration of the biomarker may be measured with an assay, for example an antibody-based method (e.g., an ELISA). As used herein, the term antibody-based method refers to any method with the use of an antibody including, but not limited to, enzyme-linked immunosorbent assay (ELISA), Western blotting, immunoprecipitation (IP), enzyme linked immunospot (ELISPOT), immunostaining, immunohistochemistry, immunocytochemistry, affinity chromatography, and the like. Preferably, an ELISA is used. The term ELISA refers to a method of detecting the presence and concentration of a biomarker in a sample, for example, before, during or after treatment with a Pt(II) selenone complex of the invention. There are several variants of ELISA, including, but not limited to, sandwich ELISA, competitive ELISA, indirect ELISA, ELISA reverse, and the like. The ELISA assay may be a singleplex assay or a multiplex assay, which refers to a type of assay that simultaneously measures multiple analytes in a single run/cycle of the assay. Preferably, a sandwich ELISA is used. The protocol for measuring the concentration of the biomarker and/or detecting the mutation in the biomarker is known to those of ordinary skill, for example by performing the steps outlined in the commercially available assay kit sold by Sigma-Aldrich, Thermo Fisher Scientific, R & D Systems, ZeptoMetrix Inc., Cayman Inc., Abcam, Trevigen, Dojindo Molecular Technologies, Biovision, and Enzo Life Sciences. The term sample includes any biological sample taken from the subject including a cell, tissue sample, or body fluid. For example, a sample may include a tumor sample, skin sample, a cheek cell sample, saliva, or blood cells. A sample can include, without limitation, a single cell, multiple cells, fragments of cells, an aliquot of a body fluid, whole blood, platelets, serum, plasma, red blood cells, white blood cells, endothelial cells, tissue biopsies, synovial fluid, and lymphatic fluid. In some embodiments, the sample is taken from a tumor. In some embodiments, the concentration of the biomarker is measured before and after the administration. When the concentration of the biomarker is maintained, the method may further comprise increasing the effective amount of at least one of the platinum(II) complex of the invention, the salt thereof, the solvate thereof, the prodrug thereof, and the combination thereof by at least 5%, at least 10%, or at least 30%, up to 50%, up to 60%, or up to 80% of an initial effective amount that is in a range of 1-100 mg/kg based on the weight of the subject. The subject may be administered with the increased dosage for a longer period (e.g. 1 week more, 2 weeks more, or 2 months more) than the duration with the initial effective amount. In some embodiments, the mutation in the biomarker is detected before administrating the composition to identify subjects predisposed to the disease. For example, women with a BRCA1 germline mutation are at a higher risk of contracting ovarian cancer. In some embodiments, the biomarkers are measured/detected after each administration. For example, the measurement may be 1-5 minutes, 1-30 minutes, 30-60 minutes, 1-2 hours, 2-12 hours, 12-24 hours, 1-2 days, 1-15 weeks, 15-20 weeks, 20-30 weeks, 30-40 weeks, 40-50 weeks, 1 year, 2 years, or any period of time in between after the administration.

Cancers/Proliferative Disorders. Cancers such as, but not limited to, sarcomas, carcinomas, melanomas, myelomas, gliomas and lymphoma (including Hodgkin lymphoma), can be treated or prevented with the platinum(II) complexes provided herein. In some embodiments, the Pt(II) complexes of the invention retain anti-cancer activity against cancer cells that are or have become resistant to conventional anti-cancer drugs such as cisplatin. When resistance develops to a conventional anticancer drug, treatment may be continued with a Pt(II) complex of the invention to which the cancer cells are sensitive.

In some embodiments, methods incorporating the use a platinum(II) complex of the present disclosure to treat or prevent cancer of the blood, brain, bladder, lung, cervix, ovary, colon, rectum, pancreas, skin, prostate gland, stomach, breast, liver, spleen, kidney, head, neck, testicle, bone, bone marrow, thyroid gland or central nervous system. In some embodiments, these methods are effective in the treatment or prevention of cervical, colon, prostate, and lung cancers. Cancers or tumor resistant to other anticancer drugs, such as cisplatin-resistant cancers, may be treated. In treating certain cancers, the best approach is often a combination of surgery, radiotherapy, and/or chemotherapy. Therefore, in at least one embodiment, the composition is employed in conjunction with conventional radiotherapy and/or chemotherapy. In another embodiment, the composition is employed with surgery. The radiotherapy and/or surgery may be before or after the composition is administered.

Other non-cancerous proliferative diseases, disorders or conditions may also be treated, such as atherosclerosis, rheumatoid arthritis, psoriasis, idiopathic pulmonary fibrosis, scleroderma, cirrhosis of the liver, or benign proliferative conditions such as verruca (warts), dermatitis, or other disorders characterized by epidermal cell proliferation.

Prostate cancer. After skin cancer, prostate cancer is the second most common cause of cancer death in American men. As shown herein, Pt(II) tetraselenone complexes exert cytotoxic and anticancer activity against prostate cancer cells. Risk factors that may be taken into account when determining whether and when to administer a treatment for prostate cancer include age with men at or above age 65 at more risk, race/ethnicity with African-American men at higher risk than Caucasian men, who are at greater risk of prostate cancer than Asian-American or Hispanic/Latino men; genetic background including presence of BRCA1 and BRCA2 mutations which increase risk as well as Lynch syndrome (hereditary non-polypsosis colorectal cancer) which is caused by inherited gene changes. Other risk or diagnostic factors include PSA (prostate specific antigen) levels in the blood and size and density of the prostate as determined by a rectal examination or by transrectal ultrasound, or pathology based on a prostate biopsy. Other symptoms may include dysuria or difficulty in urinating, blood in urine or semen, or erectile dysfunction. PET scans, CT scans or bone scans may be used to identify and monitor prostate cancer.

Prostate cancers are usually adenocarcinomas which begin in cells that produce or release mucous, however, non-adenocarcinoma forms of prostate cancer exist such as sarcomas or small-cell carcinomas. T1 and T2 stage prostate cancers are identified in the prostate, while T3 and T4 staged cancers have metastasized outside of the prostate.

Treatments for the various forms of prostate cancer, which may be administered in conjunction with administration of a Pt(II) selenone complex of the invention, include surgery (including radical prostatectomy), cryotherapy, hormone therapy, chemotherapy, immunotherapy including vaccination, targeted therapy, bone directed therapy, and radiation therapy. The Pt(II) selenone complex may be administered by itself or in combination with other therapy to a subject at risk of prostate cancer, a subject diagnosed with prostate cancer, or a subject under treatment for prostate cancer, or a subject who has already been treated (e.g., by removal of the prostate).

Ovarian carcinoma is a cancer that forms in or on an ovary. It results in abnormal cells that have the ability to invade or spread to other parts of the body. When this process begins, there may be no or only vague symptoms, however symptoms become more noticeable as the cancer progresses. These symptoms may include bloating, pelvic pain, abdominal swelling, and loss of appetite, among others. Common areas to which the cancer may spread include the lining of the abdomen, lymph nodes, lungs, and liver. About 10% of cases are related to inherited genetic risk; women with mutations in the genes BRCA1 or BRCA2 have about a 50% chance of developing the disease. The most common type of ovarian cancer, comprising more than 95% of cases, is ovarian carcinoma. There are five main subtypes of ovarian carcinoma, of which high-grade serous carcinoma is the most common. A diagnosis of ovarian cancer is usually confirmed through a biopsy of tissue, usually removed during surgery. If caught and treated in an early stage, ovarian cancer is often curable. Treatment usually includes some combination of surgery, radiation therapy, and chemotherapy. The Pt(II) selenone complex of the invention may be administered by itself or in combination with other therapy to a subject at risk of ovarian cancer, a subject diagnosed with ovarian cancer, or a subject under treatment for ovarian cancer, or a subject who has already been treated for ovarian cancer, for example, by removal of the ovaries.

Breast cancer is cancer that develops from breast tissue. Signs of breast cancer may include a lump in the breast, a change in breast shape, dimpling of the skin, fluid coming from the nipple, or a red scaly patch of skin In breast cancers with distant spread of the disease, there may be bone pain, swollen lymph nodes, shortness of breath, or yellow skin. Risk factors for developing breast cancer include being female, obesity, lack of physical exercise, drinking alcohol, hormone replacement therapy during menopause, ionizing radiation, early age at first menstruation, having children late or not at all, older age, and family history. About 5-10% of cases are due to genes inherited from a person's parents, including BRCA1 and BRCA2 among others. The Pt(II) selenone complex of the invention may be administered by itself or in combination with other therapy to a subject at risk of breast cancer, a subject diagnosed with breast cancer, or a subject under treatment for breast cancer, or a subject who has already been treated for breast cancer, for example, by removal of breast tissue.

Therapy. As used herein, the terms “therapies” and “therapy” can refer to any method, composition, and/or active ingredient that can be used in the treatment and/or management of the disease or one or more symptoms thereof. In some embodiments, the method for treating the disease involves the administration of a unit dosage or a therapeutically effective amount of the active ingredient to a subject in need thereof.

Administration. The terms “administer”, “administering”, “administration”, and the like, as used herein, refer to the methods that may be used to enable delivery of the active ingredient and/or the composition to the desired site of biological action. Routes or modes of administration are as set forth herein. These methods include, but are not limited to, oral routes, intraduodenal routes, parenteral injection including intravenous, subcutaneous, intraperitoneal, intramuscular, intravascular, or infusion, topical and rectal administration. Those of ordinary skill in the art are familiar with administration techniques that can be employed with the compounds and methods described herein. In preferred embodiments, the active ingredient and/or the composition described herein are administered orally.

The terms “effective amount”, “therapeutically effective amount”, or “pharmaceutically effective amount” refer to that amount of the active ingredient being administered which will relieve to some extent one or more of the symptoms of the disease being treated. The result can be reduction and/or alleviation of the signs, symptoms, or causes of a disease, or any other desired alteration of a biological system. For example, an “effective amount” for therapeutic uses is the amount of the platinum(II) complex of the invention, the salt thereof, the solvate thereof, the prodrug thereof, or a combination thereof as disclosed herein required to provide a clinically significant decrease in a disease. An appropriate “effective amount” may differ from one individual to another. An appropriate “effective amount” in any individual case may be determined using techniques, such as a dose escalation study.

The dosage and treatment duration are dependent on factors, such as bioavailability of a drug, administration mode, toxicity of a drug, gender, age, lifestyle, body weight, the use of other drugs and dietary supplements, the disease stage, or tolerance and resistance of the body to the administered drug, and then determined and adjusted accordingly. In at least one embodiment, the at least one of the platinum(II) complex of the invention, the salt thereof; the solvate thereof; the prodrug thereof, and the combination thereof is administered in an effective amount in a range of 1-100 mg/kg based on the weight of the subject, preferably 10-80 mg/kg, more preferably 20-50 mg/kg.

In some embodiments, a treatment will involve administering a composition comprising at least 0.5 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, 99 wt %, or 99.9 wt %, of the platinum(II) complex of the invention. The composition may comprise 0.01-50 μM, 0.01-30 μM, preferably 0.01-10 μM of the platinum(II) complex of the invention relative to the total composition. In some embodiments, the composition comprises up to 0.1 wt %, 1 wt %, 5 wt %, or 10 wt % of the pharmaceutically acceptable salt of the platinum(II) complex of the invention. In some embodiments, the composition comprises up to 0.1 wt %, 1 wt %, 5 wt %, or 10 wt % of the pharmaceutically acceptable solvate thereof of either the platinum(II) complex of the invention. These ranges include all intermediate subranges and values.

A treatment method may comprise administering a composition containing the platinum(II) complex of the invention as a single dose or multiple individual divided doses. In some embodiments, the composition is administered at various dosages (e.g., a first dose with an effective amount of 50 mg/kg and a second dose with an effective amount of 10 mg/kg). In some embodiments, the interval of time between the administration of the composition and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months, 5 months, 6 months, 7 months, 8 months, 9 months, 10 months, 11 months, 12 months, 1 year, 2 years, or any period of time in between. Preferably, the composition is administered once daily for at least 2 days, 5 days, 6 days, or 7 days. In certain embodiments, the composition and one or more additional therapies are administered less than 1 day, 1 week, 2 weeks, 3 weeks, 4 weeks, one month, 2 months, 3 months, 6 months, 1 year, 2 years, or 5 years apart.

### EMBODIMENTS

The following examples illustrate various aspects of the present invention. They are not to be construed to limit the claims in any manner whatsoever.
1. A method for treating a proliferative disease, disorder or condition in a subject comprising administering to a subject in need thereof a complex comprising a platinum atom coordinated or bound to four selenone molecules.
2. The method of embodiment 1, wherein the platinum atom is in oxidation state II and the selenone molecule used to produce the complex is selected from the group consisting of

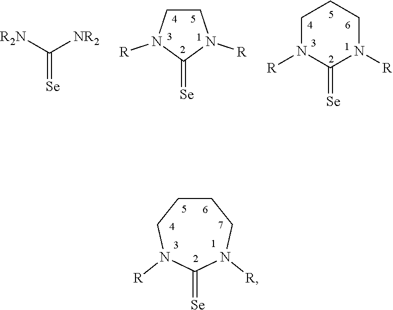

wherein the R groups are, independently, hydrogen, alkyl, aryl, halogen, haloalkyl, haloaryl, —OH, or —O-alkyl and wherein said complex comprises four selenones which may be the same or different and one or more kinds of anions. In some embodiments, the selenone moiety may be chemically modified or further substituted after its coordination or combination with a Pt(II) atom. In others, the selenone is modified prior to its use to produce the Pt(II) complex.
3. The method of embodiment 1, wherein the complex is selected from the group consisting of at least one of complex (1) [Pt(HL1)₄]Cl₂, complex (2) [Pt(HL2)₄]Cl₂, complex (3) [Pt(HL3)₄]Cl₂, complex (4) [Pt(HL4)₄]Cl₂, complex (5) [Pt(HL5)₄]Cl₂, complex (6) [Pt(HL6)₄]Cl₂ and complex (7) [Pt(HL7)₄]Cl₂, or a variant complex wherein one or two of the chloride atoms are replaced with one or more different anions.
4. The method of embodiment 1, wherein the complex comprises complex (4) [Pt(HL4)₄]Cl₂ or a variant complex wherein one or two of the chloride atoms are replaced with one or more different anions.
5. The method of embodiment 1, wherein the complex comprises complex (5) [Pt(HL5)₄]Cl₂ or a variant complex wherein one or two of the chloride atoms are replaced with one or more different anions.
6. The method of embodiment 1, wherein the proliferative disease, disorder or condition is cancer.
7. The method of embodiment 1, wherein the proliferative disease, disorder, or condition is breast cancer.
8. The method of embodiment 1, wherein the proliferative disease, disorder, or condition is ovarian cancer.
9. The method of embodiment 1, wherein the proliferative disease, disorder, or condition is prostate cancer.
10. The method of embodiment 1, wherein the proliferative disease, disorder, or condition is prostate cancer that is resistant to cisplatin.
11. A platinum(II) selenone complex comprising a platinum atom in oxidation state II and a selenone molecule selected from the group consisting of:

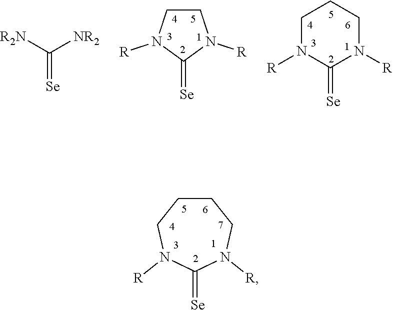

wherein the R groups are, independently, hydrogen, alkyl, aryl, halogen, haloalkyl, haloaryl, —OH, or —O-alkyl, and
wherein said complex comprises four selenones which may be the same or different and one or more kinds of anions.
12. The complex of embodiment 11 that comprises complex (1) [Pt(HL1)₄]Cl₂ or a variant complex wherein one or two of the chloride atoms are replaced with one or more different anions.
13. The complex of embodiment 11 that comprises complex (2) [Pt(HL2)₄]Cl₂ or a variant complex wherein one or two of the chloride atoms are replaced with one or more different anions.
14. The complex of embodiment 11 that comprises complex (3) [Pt(HL3)₄]Cl₂ or a variant complex wherein one or two of the chloride atoms are replaced with one or more different anions.
15. The complex of embodiment 11 that comprises complex (4) [Pt(HL4)₄]Cl₂ or a variant complex wherein one or two of the chloride atoms are replaced with one or more different anions.
16. The complex of embodiment 11 that comprises complex (5) [Pt(HL5)₄]Cl₂ or a variant complex wherein one or two of the chloride atoms are replaced with one or more different anions.
17. The complex of embodiment 11 that comprises complex (6) [Pt(HL6)₄]Cl₂ or a variant complex wherein one or two of the chloride atoms are replaced with one or more different anions.
18. The complex of embodiment 11 that comprises complex (7) [Pt(HL7)₄]Cl₂ or a variant complex wherein one or two of the chloride atoms are replaced with one or more different anions.
19. A pharmaceutical composition comprising at least one platinum(II) complex of embodiment 11 in combination with at least one pharmaceutically acceptable carrier or excipient.
20. The pharmaceutical composition of embodiment 19 that further comprises an anticancer drug, chemotherapeutic agent, or immunopotentiator.

### Example 1

### Synthesis of [Pt(selenone)₄]Cl₂ Complexes

Potassium tetrachloridoplatinate(II), K₂PtCl₄ was obtained from Strem Chemical Company, USA. Deuterated (methanol-d₄, chloroform-d₁ and dimethylsulfoxide-d₆) and other non-deuterated solvents were purchased from Sigma Aldrich or Fluka Chemical Co., and were used without further purification. The selenone ligands (HL1-HL7) were prepared according to the procedures, previously described in the literature. See F. Cristiani, F. A. Devillanova, G. Verani, J. Chem. Soc. Perkin Trans. II, 1977, 324; and M. I. M Wazeer, A. A. Isab and H. P. Perzanowski, Magn. Reson. Chem. 2003, 41, 1026-1029, each incorporated herein by reference in their entirety.

The complexes (1-7) were prepared by adding 1.0 mmol of the corresponding selenone ligand dissolved in 10 mL methanol to 0.1 g (0.25 mmol) of potassium tetrachloridoplatinate(II) dissolved in a 20 mL hot acetonitrile. The mixture was stirred under nitrogen for 1 hour using anhydrous solvents. The colored solutions were filtered and the solvents were evaporated to obtain the products. Suitable crystals of compounds 3 and 5 were obtained as yellow plates by slow evaporation at room temperature (Yield=50-60%). The analysis of these seven complexes yielded the following data:

Calculated for 1: C, 6.33; H, 2.12; N, 14.78. Found: C, 5.98; H, 2.07; N, 13.98.

Calculated for 2: C, 16.71; H, 2.80; N, 12.98. Found: C, 16.87; H, 2.86; N, 12.68.

Calculated for 3: C, 24.65; H, 4.13; N, 11.49. Found: C, 23.97; H, 4.08; N, 11.23.

Calculated for 4: C, 27.97; H, 4.69; N, 10.87. Found: C, 25.90; H, 4.42; N, 10.02.

Calculated for 5: C, 27.97; H, 4.69; N, 10.87. Found: C, 27.05; H, 4.57; N, 10.33.

Calculated for 6: C, 20.92; H, 4.51; N, 12.20. Found: C, 2036; H, 3.58; N, 12.14.

Calculated for 7: C, 24.65; H, 4.13; N, 11.49. Found: C, 23.78; H, 4.02; N, 10.97.

### X-ray Structure Determinations

The intensity data were collected at 203K (−70° C.) on a Stoe Mark II-Image Plate Diffraction System equipped with a two-circle goniometer using MoKα graphite monochromated radiation (λ=0.71073 Å). See Stoe& Cie. X-Area & X-RED32. Stoe & Cie GmbH, Darmstadt, Germany. 2009, incorporated herein by reference in its entirety. The structures were solved by direct methods with SHELX-97. See G. M. Sheldrick, Acta Cryst., 2008, A64, 112-122, incorporated herein by reference in its entirety. The refinement and all further calculations were carried with SHELX-2014. See G. M. Sheldrick. Alda Cryst., 2015, C71, 3-8, incorporated herein by reference in its entirety. The N—H H atoms were located in a difference Fourier map and refined with distance restraints: N—H=0.87(2) Å with 1.2U_{eq}(N). The C-bound H-atoms were included in the calculated positions and treated as riding atoms: C—H=0.97-0.98 Å with Uᵢₛₒ(H)=1.5U_{eq}(C) for methyl H atoms and =1.2U_{eq}(C) for other H-atoms. The non-H atoms were refined anisotropically, using weighted full-matrix least-squares on F². A semi-empirical absorption correction was applied using the MULABS routine in PLATON. See A. L. Spek. Acta Cryst., 2009, D65, 148-155, incorporated herein by reference in its entirety. The figures were drawn using the Mercury program. See C. F. Macrae, I. J. Bruno, J. A. Chisholm, R. Edgington, P. McCabe, E Pidcock, L. Rodriguez-Monge, R. Taylor, J. van de. Streek, P. A. Wood, J. Appl. Cryst., 2008, 41, 466-470, incorporated herein by reference in its entirety. The crystal data and details of refinement are given in Table 1.

TABLE 1
Crystal data and structure refinement details for compounds 3 and 5

| Compound | 3 | 5 |
|---|---|---|
| CCDC deposit no. | 1454933 | 1448338 |
| Chemical formula | [C₂₀H₄₀N₈Se₄Pt]²⁺ • 2(Cl)⁻ | [C₂₄H₄₈N₈Se₄Pt]²⁺ • 2(Cl)⁻ |
| Molecular weight | 974.43 | 1030.53 |
| Crystal system, space group | Triclinic, P-1 | Monoclinic, P2₁/c |
| Temperature (K) | 203 | 190 |
| a, b, c (Å) | 9.2384 (9), 9.6001 | 11.6644 (6), 25.9727 (17), |
| | (10), 10.7454 (12) | 13.5261 (8) |
| α, β, γ (°) | 116.430 (8), 96.169 (9), | 90, 98.711 (5), 90 |
| | 109.714 (8) | |
| V (Å3) | 764.95 (15) | 4050.5 (4) |
| Z | 1 | 4 |
| μ (mm-1) | 9.54 | 7.22 |
| Crystal size (mm) | 0.40 × 0.24 × 0.03 | 0.40 × 0.40 × 0.40 |
| Absorption correction | Multi-scan | Multi-scan |
| Tmin, Tmax | 0.598, 1.000 | 0.773, 1.000 |
| No. of measured, independent, | 11226, 3088, 2360 | 18780, 9348, 6857 |
| | (Rint = 0.105) | (Rint = 0.045) |
| observed [I > 2σ(I)] reflections | | |
| (sin θ/λ)max (Å-1) | 0.622 | 0.685 |
| R[F2 > 2σ(F2)], wR(F2), S | 0.042, 0.058, 1.16 | 0.064, 0.200, 1.06 |
| No. of reflections | 3088 | 9348 |
| No. of parameters | 168 | 361 |
| Largest diff. Peak and hole | 1.35, −2.19 | 4.26, −1.71 |
| (e Å-3)Δρmax, Δρmin (e Å-3) | | |

### IR and NMR Measurements

The FTIR spectra of the ligands and their platinum(II) complexes were carried out on a Nicolet 6700 FTIR spectrophotometer using KBr pellets in the range between 4000 to 400 cm⁻¹. The ¹H, ¹³C and ⁷⁷Se NMR spectra were recorded in DMSO-d₆ on a Jeol JNM 500 NMR spectrophotometer operating at 500.01, 125.65 and 95.35 MHz respectively. The spectral conditions were; 32 K data points, 0.963 s acquisition time, 3.2 s pulse delay and a 5.75 μs pulse width for ¹H NMR, and 32 K data points, 0.963 s acquisition time, 2.0 s pulse delay and a 5.12 μs pulse width for ¹³C NMR. The chemical shifts were measured relative to TMS. The ⁷⁷Se NMR chemical shifts were recorded relative to external reference (NaHSeO₃ in D₂O) at 1308.00 ppm, using 2.0 s pulse delay and using 0.311 s acquisition time. The ¹⁹⁵Pt NMR spectra were recorded in methanol-d₄ using a Varian 400 MHz NMR spectrometer at 298 K. The spectra were referenced with respect to potassium tetrachloridoplatinate(II) in D₂O at 0 ppm.

K₂PtCl₄ and selenones (HLₙ) were mixed in a 1:4 molar ratio in methanol-acetonitrile medium. The composition obtained by elemental analysis corresponds to the general formula of the complexes, [Pt(HL)₄]Cl₂. Table 2 lists the significant IR bands of free selenones and their platinum(II) complexes. The v(C═Se) vibration, which occurs around 600 cm⁻¹ for free ligands shifts towards a lower frequency upon complexation as observed for other selenone complexes. See S. Ahmad, A. A. Isab, A. R. Al-Arfaj and A. P. Arnold, Polyhedron, 2002, 21, 2099-2105; S. Ahmad, A. A. Isab, Inorg. Chem. Commun., 2002, 5, 355-357; S. Ahmad, A. A. Isab, A. P. Arnold, J. Coord. Chem., 2003, 56, 539-544; and A. A. Isab, M. I. M. Wazeer, M. Fettouhi, S. Ahmad, W. Ashraf, Polyhedron, 2006, 25, 2629-2636, each incorporated herein by reference in their entirety. The v(N—H) and v(C—N) bands appear around 3200 cm⁻¹ and 1500 cm⁻¹ respectively.

Upon coordination, these bands shift to higher wave numbers with some exceptions. A low frequency shift in the v(C═Se) band and a high frequency shift in the v(N—H) and v(C—N) bands in the complexes compared to free ligands indicate the existence of selenone forms of the ligands in the solid state. The absorptions in the range of 300-275 cm⁻¹ in the Far IR region were attributed to the v(Pt—Se) vibrations. See D. Fregona et al.

TABLE 2
Selected IR absorptions (cm⁻¹) of the
selenones and their Platinum(II) complexes

| | | IR frequencies (cm⁻¹) | | | | |
|---|---|---|---|---|---|---|
| | Species | v(C═Se) | v(C—N) | v(N—H) | v(Pt—Se) | |
| NMR | HL1 | 736 | 1520 | 3265 | | Studies |
| | 1 | 586 | 1609 | 3310 | 300 | |
| | HL2 | 561 | 1463 | 3250 | | |
| | 2 | 566 | 1520 | 3369 | 250 | |
| | HL3 | 514 | 1465 | 3198 | | |
| | 3 | 574 | 1505 | 3106 | 285 | |
| | HL4 | 513 | 1460 | 3210 | | |
| | 4 | 501 | 1510 | 3390 | 300 | |
| | HL5 | 601 | 1453 | 3210 | | |
| | 5 | 598 | 1532 | 3304 | 281 | |
| | HL6 | 601 | 1430 | 3200 | | |
| | 6 | 587 | 1473 | 3285 | 290 | |
| | HL7 | 615 | 1453 | 3224 | | |
| | 7 | 606 | 1549 | 3386 | 275 | |

In solution, the complexes were characterized by ¹H, ¹³C, ⁷⁷Se and ¹⁹⁵Pt NMR in DMSO-d₆, and CD₃OD respectively. The ¹H NMR chemical shifts of N—H protons and the ¹³C NMR chemical shifts of all carbon atoms of ligands and the complexes are given in Table 3. In ¹H NMR spectra of the complexes, the N—H signal of selenones shifted downfield by more than 1.0 ppm from its position in free ligands. The deshielding is related to an increase in TC character of the C—N bond upon coordination. In ¹³C NMR spectra, the >C═Se resonance of selenones is shifted upfield upon complexation as compared to the free positions (Table 4) in accordance with the data observed for other complexes of selenones. See S. Ahmad, A. A. Isab, A. R. Al-Arfaj and A. P. Arnold, Polyhedron, 2002, 21, 2099-2105; S. Ahmad, A. A. Isab, Inorg. Chem. Commun., 2002, 5, 355-357; S. Ahmad, A. A. Isab, A. P. Arnold, J. Coord. Chem., 2003, 56, 539-544; and A. A. Isab et al.

A shift of about 8 to 10 ppm in C-2 resonance (except for complex 3, where it is 2.75 ppm) indicates that in all the complexes, the selenone ligands are coordinated to platinum(II) through the selenium atom. A small shift in other resonances shows that nitrogen atoms are not involved in coordination. The binding through nitrogen has been reported in the platinum(II) complex of selenourea dianion. See W. Henderson, B. K. Nicholson, M. B. Dinger, Inorg. Chim. Acta, 2003, 355, 428-431, incorporated herein by reference in its entirety. A deshielding effect at C-4 (or the carbon atom at its equivalent position) is due to an increase in ncharacter of the C—N bond. It can be seen from Table 4 that as the ring size of the ligand is increased the shift difference at >C═Se resonance also increases. These values reflect that the platinum(II) complex formed by diazepane-2-thione (HL7) would be more stable than those formed by imidazolidine-2-thione (HL2) and diazinane-2-thione (HL6) ligands. A similar trend was observed for mercury(II) complexes of selenones. See A. A. Isab et al.

TABLE 3
¹H and ¹³C{¹H} NMR chemical shifts
of the Pt(II) complexes with selenones in DMSO

| Species | N—H | C-2 | C-4 | C-5 | C-6 | C-7 | N—C1 | N—C2 | CH₃ |
|---|---|---|---|---|---|---|---|---|---|
| HL1 | 7.59 | 178.83 | — | — | — | — | — | — | — |
| 1 | 8.55 | 171.19 | — | — | — | — | — | — | — |
| | 0.96 | −7.64 | — | — | — | — | — | — | — |
| HL2 | 8.33 | 177.08 | 44.94 | 44.94 | — | — | — | — | — |
| 2 | 9.59 | 168.88 | 45.34 | 46.34 | — | — | — | — | — |
| | 1.27 | −8.20 | 0.40 | 0.40 | — | — | — | — | — |
| HL3 | 8.32 | 178.66 | 43.33 | 47.91 | — | — | 42.51 | — | 12.09 |
| 3 | 9.67 | 175.91 | 43.18 | 48.96 | — | — | 43.18 | — | 12.23 |
| | 1.35 | −2.75 | −0.15 | 1.05 | — | — | 0.67 | — | 0.14 |
| HL4 | 8.81 | 179.55^{a} | 50.19 | 48.62 | — | — | 42.60 | 10.99 | 10.99 |
| 4 | 9.66 | 170.42 | 49.82 | 49.59 | — | — | 43.82 | 20.76 | 11.1 |
| | 0.85 | −9.13 | −0.37 | 0.97 | — | — | 1.22 | 0.39 | 0.11 |
| HL5 | 8.26 | 177.44^{a} | 42.65 | 42.69 | — | — | 48.21 | — | 19.45 |
| 5 | 9.65 | 167.36 | 43.13 | 43.69 | — | — | 48.92 | — | 19.41 |
| | 1.39 | −10.41 | 0.48 | 1.00 | — | — | 0.71 | — | −0.04 |
| HL6 | 8.13 | 169.14^{b} | 40.10 | 18.80 | 40.10 | — | — | — | — |
| 6 | 9.16 | 164.77 | 40.15 | 18.86 | 40.15 | — | — | — | — |
| | 1.03 | −8.34 | 0.05 | 0.06 | 0.05 | — | — | — | — |
| HL7 | 8.07 | 180.83^{b} | 45.5 | 26.86 | 26.86 | 45.5 | — | — | — |
| 7 | 9.14 | 171.68 | 46.64 | 26.25 | 26.25 | 46.64 | — | — | — |
| | 1.07 | −9.15 | 1.14 | −0.61 | −0.61 | 1.14 | — | — | — |

^{a}in CDCl₃,
^{b}in D₂O

The ⁷⁷Se NMR spectroscopy seems to be the most effective technique for characterizing the complexes of selenium donor ligands because in ⁷⁷Se NMR spectra a large upfield shift is observed for the ligands upon complexation. See A. A. Isab et al.; W. Henderson et al.; and H. Amouri, J. Moussa, A. K. Renfrew, P. J. Dyson, M. N. Rager, L.-M. Chamoreau, Angew. Chem. Int. Ed., 2010, 49, 7530-7533, each incorporated herein by reference in their entirety. Table 4 shows that the selenium resonances are shifted upfield by about 4-71 ppm upon coordination. This very large shielding provides a clear evidence for selenium binding to the metal center. The complex 6 shows the highest shift difference of 71 ppm. This trend is not consistent with the ¹³C NMR data, where the HL7 complex shows the greatest difference.

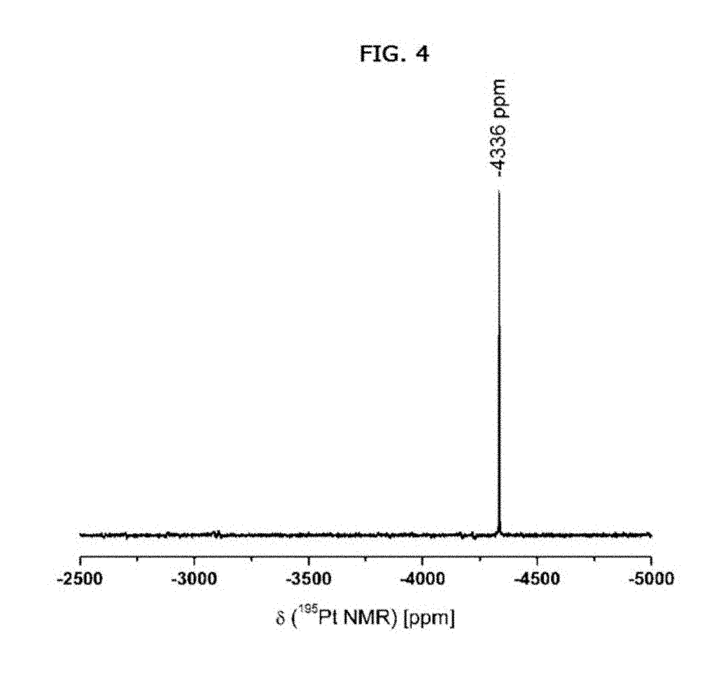

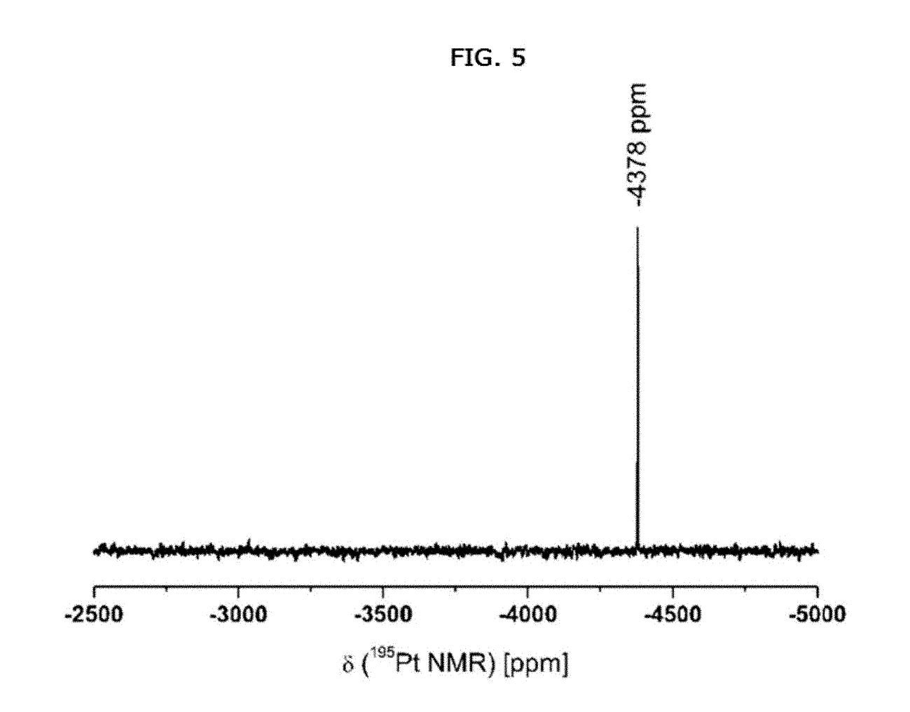

The ¹⁹⁵Pt NMR spectroscopy was also employed for thorough characterization of the prepared complexes, as the number of signals and their chemical shift in the ¹⁹⁵Pt NMR spectra provide efficient evidence about the purity and, also generally, the coordination environment of the metal. The spectra of complexes 1 and 2 could not be collected due to insufficient solubility in methanol-d₄ as well as in deuterated N,N-dimethylformamide required for these experiments. The spectra of the platinum(II) complexes showed a single resonance in the region from −4314 ppm to −4378 ppm (Table 4), which is in agreement with the previously reported related species. See N. R. Champness, W. Levason, J. J. Quirk, G. Reid, Polyhedron, 1995, 14, 2753-2758; and W. Levason, M. Nirwan, R. Ratnani, G. Reid, N. Tsoureas, M. Webster, Dalton Trans., 2007, 439-444, each incorporated herein by reference in their entirety. These results confirmed the proposed composition of the platinum(II) complexes and the homogenous coordination sphere around the central atoms. For two representative examples, depicting the ¹⁹⁵Pt NMR spectra of the complexes 3 and 7, see FIG. 4 and FIG. 5.

TABLE 4
⁷⁷Se{¹H} and ¹⁹⁵Pt NMR chemical shifts (in ppm) of the
Pt(II) complexes (1-7) in DMSO-d₆, and CD₃OD
respectively (upfield shifts are denoted by Δ)

| Species | (δ)⁷⁷Se | (δ)¹⁹⁵Pt |
|---|---|---|
| (HL1) | 200.70 | — |
| [Pt(HL1)₄]Cl₂ | 172.84 | N/A |
| Δ | −27.88 | — |
| HL2 | 73.53 | — |
| [Pt(HL2)₄]Cl₂ | 60.61 | N/A |
| Δ | −12.92 | — |
| HL3 | 73.53 | — |
| [Pt(HL3)₄]Cl₂ | 60.61 | −4336 |
| Δ | −12.92 | |
| HL4 | 57.93 | — |
| [Pt(HL4)₄]Cl₂ | 43.70 | −4314 |
| Δ | −14.23 | |
| HL5 | 69.29 | — |
| [Pt(HL5)₄]Cl₂ | 64.91 | −4318 |
| Δ | −4.38 | |
| HL6 | 199.93 | — |
| [Pt(HL6)₄]Cl₂ | 176.69 | −4220 |
| Δ | −23.24 | |
| HL7 | 292.00 | — |
| [Pt(HL7)₄]Cl₂ | 273.78 | −4378 |
| Δ | −18.22 | |

### Description of Crystal Structures

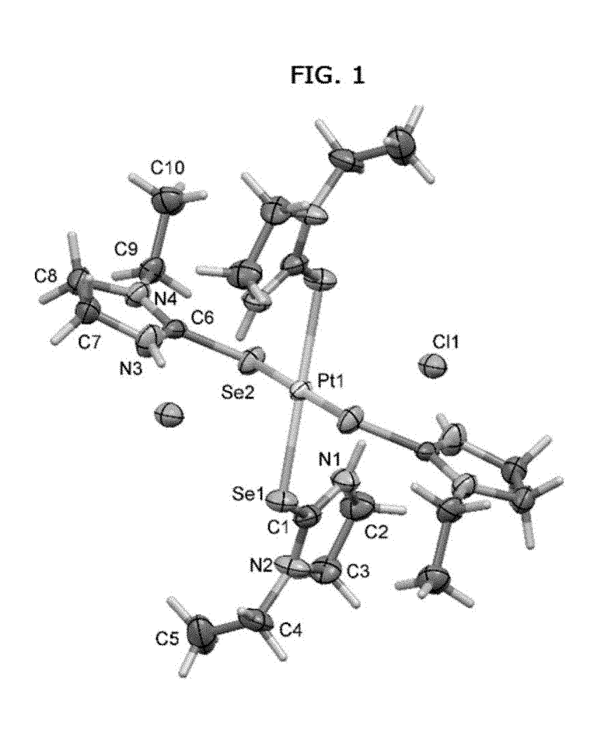

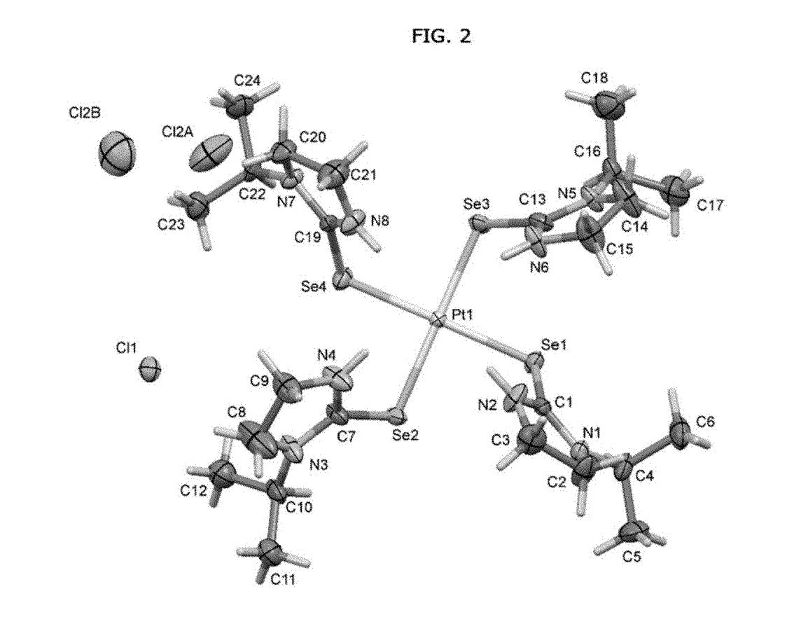

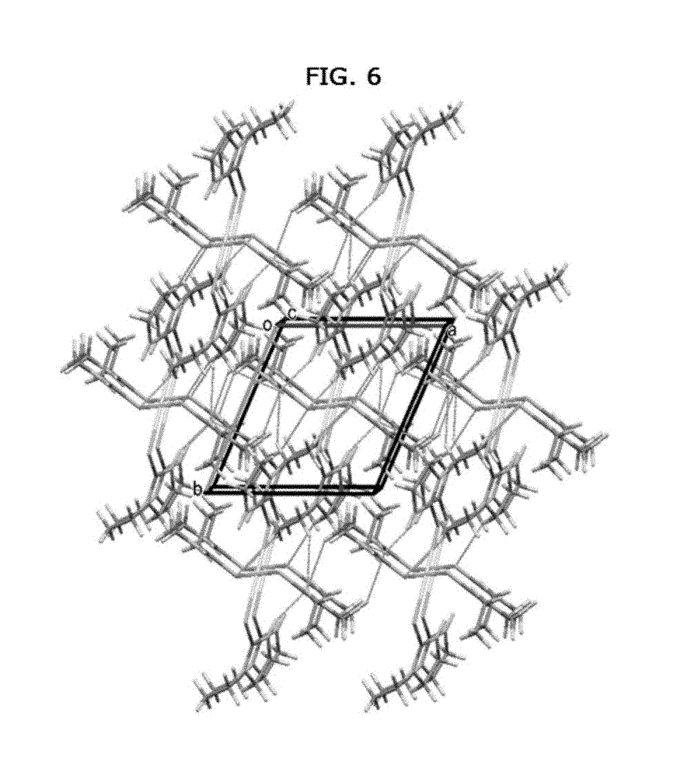

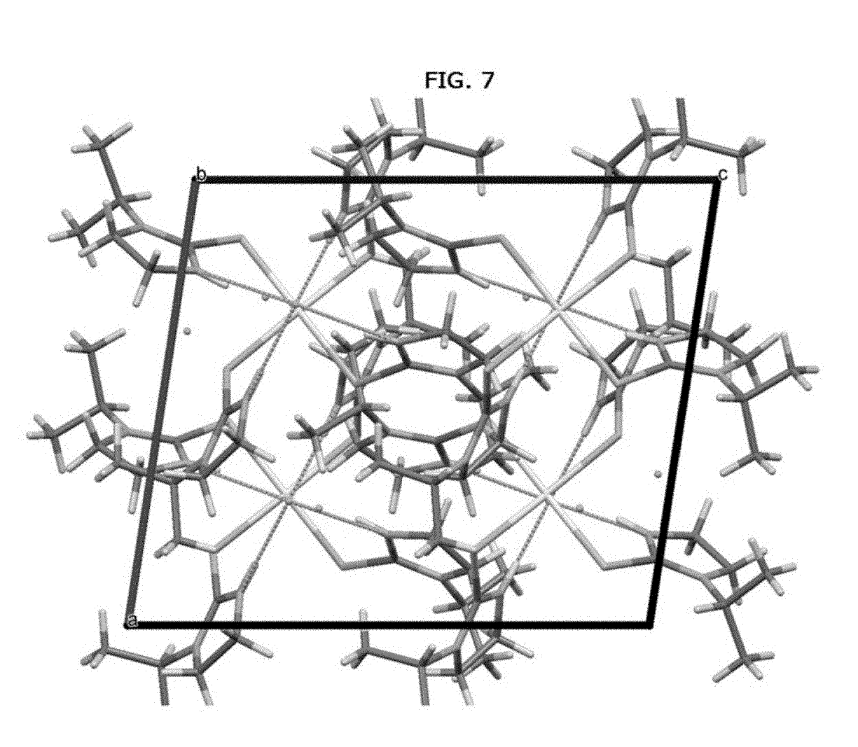

The X-ray structures of compounds 3 and 5 are shown in FIGS. 1, and 2, respectively. The geometrical parameters are given in Table 5. The Pt(II) atoms in both 3 and 5 are coordinated to four selenium atoms, each belonging to an N-alkylimidazolidine-2-selenone ligand. The Pt—Se bond lengths of 2.4200(11)-2.4389(7) Å are similar to the related compounds. See J. Moussa et al.; M. M. Kubicki et al; and W. Henderson. In 3, the cis Se—Pt—Se angles are 86.98(3)° and 93.02(3)°, while the trans angles are 180°. In 5, the cis angles around Pt are nearly 90°, whereas the trans angles are 165.64(4)° and 173.49(4)° (Table 6). These values reflect that the geometry at platinum is somewhat distorted square planar. The SeCN₂ moieties of the ligand molecules are essentially planar. The smaller N—C(Se) bond lengths compared to the other N—C bond distances are in agreement with a marked double bond π-character in the N—C(Se) bond. In 3, the N—H groups (N1-H1 and N3-H3) of two cis selenone ligands are engaged in hydrogen bonding with a common chloride ion giving a hydrogen bonding bridge [N—H . . . Cl . . . H—N] as shown in FIG. 6. A closer look to the hydrogen bonding interactions in 5 reveals that all four selenone ligands are engaged in hydrogen bonding with one chloride counter ion resulting in an umbrella like structure as shown in FIG. 7.

This H-bonding scheme gives two decametallacycles [PtSeCNH . . . Cl . . . HNCS] in which all the selenium atoms are pushed out of the [PtSe₄] mean plane. The details of hydrogen-bond geometry (Å, °) in 3 and 5 are given in Table 5.

TABLE 6
Hydrogen-bond geometry (Å, °) of compound 3

| D-H . . . A | D-H | H . . . A | D . . . A | D-H . . . A |
|---|---|---|---|---|
| N1—H1N . . . Cl1 | 0.88 (2) | 2.28 (3) | 3.122 (6) | 159 (6) |
| N3—H3N . . . Cl1ⁱ | 0.87 (2) | 2.33 (3) | 3.174 (7) | 165 (6) |
| C2—H2A . . . Cl1ⁱⁱ | 0.98 | 2.99 | 3.715 (7) | 132 |
| C3—H3A . . . Se2ⁱⁱⁱ | 0.98 | 3.04 | 3.958 (8) | 157 |
| C3—H3B . . . Cl1ⁱⁱ | 0.98 | 2.86 | 3.625 (8) | 135 |
| C5—H5B . . . Se2^{iv} | 0.97 | 3.08 | 4.045 (8) | 171 |
| C8—H8A . . . Sc1^{v} | 0.98 | 3.14 | 3.865 (7) | 132 |
| C8—H8A . . . Cl1^{vi} | 0.98 | 2.88 | 3.707 (7) | 143 |

Symmetry codes: (i)−x+1, −y+1, −z+1; (ii) −x+2, −y+2, −z+2; (iii) −x+1, −y+2, −z+2; (iv) x, y+1, z; (v) −x, −y+1, −z+1; (vi) x−1, y, z.

### Interactions of the Selected Complexes with L-cysteine and Reduced Glutathione by Mass Spectrometry

In order to describe the behavior of complexes in different media, i.e. to better understand their stability in protogenic media and interactions of the complexes with sulfur-containing biomolecules under physiological conditions, mass spectrometric experiments involving the interacting systems, containing the mixture of L-cysteine and reduced L-glutathione, were performed.

The electrospray-ionization mass spectrometry (ESI-MS) was used to perform the interaction experiments of the selected two cytotoxic platinum(II) complexes 4 and 5 using the ThermoLCQ Fleet Ion Trap mass spectrometer, in positive ionization mode. The complexes were dissolved in methanol at the final concentration of 10 μM with the physiological concentrations of L-cysteine and reduced glutathione (dissolved in water) at the final concentration of 290 μM, and 6 μM, respectively). See G. Salemi, M. C. Gueli, M. D'Amelio, V. Saia, P. Mangiapane, P. Aridon, P. Ragonese and I. Lupo, Neural. Sci., 2009, 30, 361-364, incorporated herein by reference in its entirety. The measured solutions were injected into the mass spectrometer using the HPLC autosampler (Ultimate 3000, Dionex) in 10 μL spikes. The mass spectra were recorded in the range of 50-1400 m/z. No additional tuning was needed to perform the analyses.

The stability in methanol:water (1:1, v/v) solutions as compared to the pure methanol and interactions of complexes in methanol:water (1:1, v/v)solutions containing the complexes 4 and 5 at the final concentrations of 10 μM and the physiological levels of L-cysteine (Cys) and L-glutathione (GSH), at 290, and 6 μM concentration, respectively, were measured by ESI+MS immediately after preparation and 24 h after preparation. See G. Salemi et al. The time-dependent changes in the spectra were significant. The spectra measured immediately after preparation contained mostly the ionic species similar to those identified in reference methanol solutions. On the other hand, 24 h after preparation, the mass spectra contained a rich variety of ionic species originating either from the hydrolysis or coordination and other interactions of cysteine and reduced glutathione. These results indicate that the ligand exchange reactions are relatively rapid.

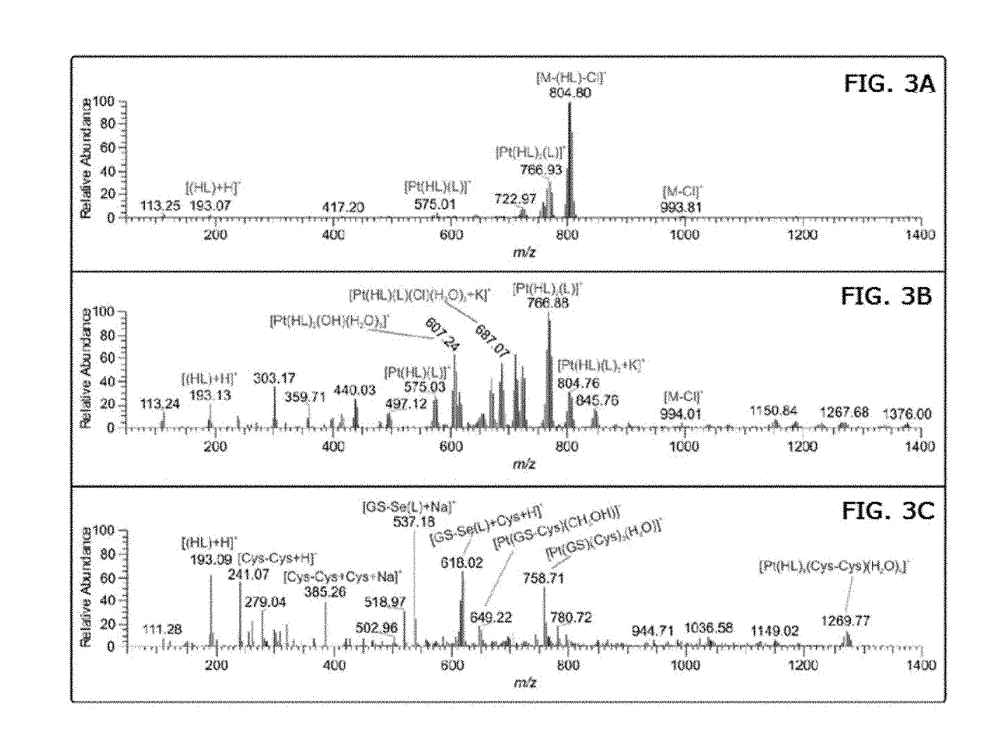

In the reference mass spectra obtained for the methanol solution, the following species were identified; see FIG. 3A: [HL+H]⁺ at 193.07 m/z; [Pt(HL)L]⁺ at 575.01 m/z; [Pt(HL)₂L]⁺ at 766.93 m/z; [M-(HL)-Cl]⁺ at 804.80 m/z; and [M-Cl]⁺ at 993.8 m/z.

In the mass spectra of the complexes containing HL4, and HL5 ligands (4 and 5) respectively, in the methanol:water mixture (1:1, v/v), the following types of ionic species were identified (See FIG. 3B): [(HL)+H]⁺ at 193.13 m/z; [Pt(HL)L]⁺ at 575.03 m/z; [Pt(HL)₂(OH)(H₂O)₃]⁺ at 607.24 m/z; [Pt(HL)(L)(Cl)(H₂O)₂+K]⁺ at 687.07 m/z; [Pt(HL)₂L]⁺ at 766.88 m/z; [Pt(HL)(L)₂+K]⁺ at 804.76 m/z; and [M-Cl]⁺ at 994.01 m/z.

All mass spectra of the interacting mixtures of the complexes with L-cysteine and reduced glutathione showed analogical new species, involving both sulfur-containing biomolecules either coordinated to platinum or forming the recombination pseudomolecules and oxidized species.

The following species, confirming the ability of the complexes to interact with the sulfur-containing molecules were identified within the mass spectra of the interacting systems; see FIG. 3C): [(HL)+H]⁺ at 193.13 m/z; [Cys-Cys+H]⁺ at 241.07 m/z; [Cys-Cys+Cys+Na]⁺ at 385.26 m/z; [GS-Se(L)+Na]⁺ at 537.18 m/z; [GS-Se(L)+Cys+H]⁺ at 618.02 m/z; [Pt(GS-Cys)+CH₃OH]⁺ at 649.22 m/z; [Pt(GS)(Cys)₂(H₂O)]⁺ at 785.71 m/z; and [Pt(HL)₄(Cys-Cys)+(H₂O)₄]⁺ at 1269.77 m/z.

### Example 2

### Assessment of Cytotoxicity In Vitro

To determine the biological potential of the prepared compounds, complexes 1-7 were evaluated for in vitro cytotoxicity by the MTT assay against human cancer cells of ovarian carcinoma (A2780), ovarian carcinoma resistant to cisplatin (A2780R), human breast adenocarcinoma (MCF-7), and prostate carcinoma (22Rv1).

In vitro cytotoxicity was evaluated by the MTT assay (MTT=3-(4,5-dimethylthiazol-2-yl)-2,5-diphenyltetrazolium bromide) against human breast adenocarcinoma (MCF7; ECACC No. 86012803), ovarian carcinoma (A2780; ECACC No. 93112517), ovarian carcinoma resistant to cisplatin (A2780R; ECACC No. 93112519), and prostate carcinoma (22Rv1; ECACC No. 105092802) cell lines. All the human cancer cell lines were purchased from European Collection of Cell Cultures (ECACC). The cells were cultivated according to the manufacturer's manuals and maintained in an atmosphere containing 5% CO₂ in a humidified incubator at 37° C. The experimental procedure was performed according to the literature. See R. Křikavová, J. Hošek, J. Vančo, J. Hutyra, Z. Dvořák, Z. Trávniček, PLoS One, 2014, 9(9), e107373, incorporated herein by reference in its entirety. All the experiments were conducted in triplicate. The results were expressed as ICso values along with standard deviations (SD). The significance of the differences between the compared groups of results was assessed by the ANOVA analysis, with p<0.05 considered to be significant (QC Expert 3.2, Statistical software, TriloByte Ltd.). See QC Expert 3.2, Statistical software, TriloByte Ltd., Pardubice, Czech Republic. 2009, incorporated herein by reference in its entirety. The results of cytotoxicity against the selected cancer cells were referenced to the clinically used chemotherapeutic drug cisplatin (Table 5).

TABLE 5
In vitro cytotoxicity of platinum complexes 1-7 and cisplatin given as IC₅₀ ± S.D. in μM.

| Compound* | A2780 | A2780R | 22RvI | MCF7 |
|---|---|---|---|---|
| Cisplatin | 26.8 ± 2.6 | >50 | 17.6 ± 4.5 | 39.6 ± 1.8 |
| 2 | >50 | >50 | >50 | >50 |
| 3 | >50 | >50 | >50 | >50 |
| 4 | 44.7 ± 1.2 | >50 | 46.2 ± 3.8 | >50 |
| 5 | 30.8 ± 0.9 | >50 | >50 | >50 |
| 6 | >50 | >50 | >50 | >50 |
| 7 | >50 | >50 | >50 | >50 |

*The complex 1 could not be tested owing to the instability of the sample under the testing conditions. Owing to low solubility in the media used the complexes were tested up to the concentration of 50 μM only.

Two complexes 4 and 5, involving the N-propylimidazolidine-2-selenone and N-isopropylimidazolidine-2-selenone ligands, were effective against the A2780 cells (IC₅₀=44.7 μM and 30.8 μM, respectively) on the comparable level as cisplatin (IC₅₀=26.8 μM). The complex 4 also showed some cytotoxicity against 22Rv1 cells (see Table 5). The platinum(II) complexes with other selenium-containing ligands (dimethylpyrazole-based selenium ligands, and selenocarbazones) were also found to exhibit poor cytotoxicity. See S. M. Chopade et al.; and N. Gligorijević, T. Todorović, S. Radulović, D. Sladić, N. Filipović, D. Godevac, D. Jeremić, K. Andelković, Eur. J. Med. Chem., 2009, 44, 1623-1629, each incorporated herein by reference in their entirety.

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the words “a” and “an” and the like carry the meaning of “one or more” unless the context clearly indicates otherwise.

Within the description of this disclosure, where a numerical limit or range is stated, the endpoints are included unless stated otherwise. Also, all values and subranges within a numerical limit or range are specifically included as if explicitly written out. For example, a range of 0 to 10 wt % includes 0. 0.25, 0.5, 1, 1.5, 2, 2.5, 3, 4, 5, 6, 7, 8, 9, 9.75, 9.99, <10, and 10.

The terms “including”, “such as”, “for example” and the like not intended to limit the scope of the present disclosure. They generally refer to one or more elements falling with a class or genus of other similar elements.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by insertion of a space or underlined space into a link, for example, before “www” or after “II” and may be reactivated by removal of the space.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all subranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it is also envisioned that Parameter X may have other ranges of values including 1-9, 2-9, 3-8, 1-8, 1-3, 1-2, 2-10, 2.5-7.8, 2-8, 2-3, 3-10, and 3-9, as mere examples.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for treating a proliferative disease, disorder, or condition comprising administering to a subject in need thereof a complex comprising a platinum atom coordinated or bound to four selenone molecules:
wherein the platinum atom is in oxidation state II and the selenone molecule used to produce the complex is selected from the group consisting of: 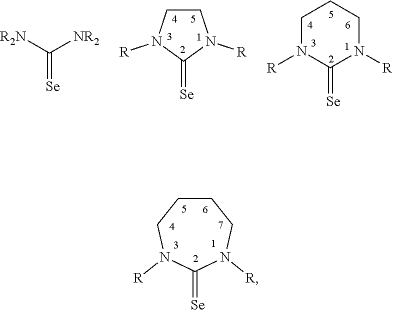
wherein the R groups are, independently, hydrogen, alkyl, aryl, halogen, haloalkyl, haloaryl, —OH, or —O-alkyl and wherein said complex comprises four selenones which may be the same or different and one or more kinds of anions; and
wherein the complex is selected from the group consisting of at least one of complex (1) [Pt(HL1)₄]Cl₂, complex (2) [Pt(HL2)₄]Cl₂, complex (3) [Pt(HL3)₄]Cl₂ complex (4) [Pt(HL4)₄]Cl₂, complex (5) [Pt(HL5)₄]Cl₂ complex (6) [Pt(HL6)₄]Cl₂ and complex (7) [Pt(HL7)₄]Cl₂, or a variant complex wherein one or two of the chloride atoms are replaced with one or more different anions.

2. The method of claim 1, wherein the platinum atom is in oxidation state II and the selenone molecule used to produce the complex is: 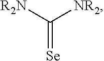
wherein the R groups are, independently, hydrogen, alkyl, aryl, halogen, haloalkyl, haloaryl, —OH, or —O-alkyl and wherein said complex comprises four selenones which may be the same or different and one or more kinds of anions.

3. The method of claim 1, wherein the complex is complex (1) [Pt(HL1)₄]Cl₂, or a variant complex wherein one or two of the chloride atoms are replaced with one or more different anions.

4. The method of claim 1, wherein the complex comprises complex (4) [Pt(HL4)₄]Cl₂ or a variant complex wherein one or two of the chloride atoms are replaced with one or more different anions.

5. The method of claim 1, wherein the complex comprises complex (5) [Pt(HL5)₄]Cl₂ or a variant complex wherein one or two of the chloride atoms are replaced with one or more different anions.

6. The method of claim 1, wherein the proliferative disease, disorder or condition is cancer.

7. The method of claim 1, wherein the proliferative disease, disorder, or condition is breast cancer.

8. The method of claim 1, wherein the proliferative disease, disorder, or condition is ovarian cancer.

9. The method of claim 1, wherein the proliferative disease, disorder, or condition is prostate cancer.

10. The method of claim 1, wherein the proliferative disease, disorder, or condition is prostate cancer that is resistant to cisplatin.

11. A platinum(II) selenone complex comprising a platinum atom in oxidation state II and a selenone molecule selected from the group consisting of: 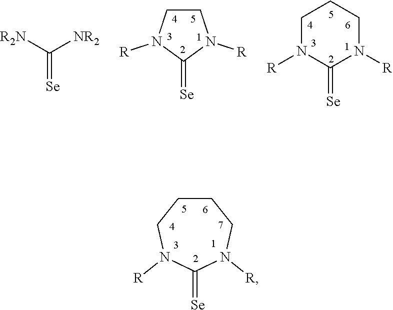
wherein the R groups are, independently, hydrogen, alkyl, aryl, halogen, haloalkyl, haloaryl, —OH, or —O-alkyl and
wherein said complex comprises four selenones which may be the same or different and one or more kinds of anions.

12. The complex of claim 11 that comprises complex (1) [Pt(HL1)₄]Cl₂ or a variant complex wherein one or two of the chloride atoms are replaced with one or more different anions.

13. The complex of claim 11 that comprises complex (2) [Pt(HL2)₄]Cl₂ or a variant complex wherein one or two of the chloride atoms are replaced with one or more different anions.

14. The complex of claim 11 that comprises complex (3) [Pt(HL3)₄]Cl₂ or a variant complex wherein one or two of the chloride atoms are replaced with one or more different anions.

15. The complex of claim 11 that comprises complex (4) [Pt(HL4)₄]Cl₂ or a variant complex wherein one or two of the chloride atoms are replaced with one or more different anions.

16. The complex of claim 11 that comprises complex (5) [Pt(HL5)₄]Cl₂ or a variant complex wherein one or two of the chloride atoms are replaced with one or more different anions.

17. The complex of claim 11 that comprises complex (6) [Pt(HL6)₄]Cl₂ or a variant complex wherein one or two of the chloride atoms are replaced with one or more different anions.

18. The complex of claim 11 that comprises complex (7) [Pt(HL7)₄]Cl₂ or a variant complex wherein one or two of the chloride atoms are replaced with one or more different anions.

19. A pharmaceutical composition comprising at least one platinum(II) complex of claim 11 in combination with at least one pharmaceutically acceptable carrier or excipient, or in combination with at least one carrier or excipient and an anticancer drug, chemotherapeutic agent, or immunopotentiator.

20. (canceled)

21. A method for treating ovarian carcinoma comprising:
administering to a subject in need thereof an active ingredient consisting essentially of a complex comprising a platinum atom coordinated or bound to four selenone molecules; wherein said complex comprises complex (1) [Pt(HL1)₄]Cl₂; where HL1 is: [chemical structure]
